SEQUENCE LISTING (2 sequences)

SEQ ID NO: 1 - protein, 4 aa, Artificial Sequence
  caspase-1 specific fluorogenic peptide substrate
YVAD

SEQ ID NO: 2 - protein, 4 aa, Artificial Sequence
  caspase-3 specific fluorogenic peptide substrate
DEVD

(19) United States
(12) Patent Application Publication
(10) Pub. No.: US 2003/0044776 A1
(43) Pub. Date: Mar. 6, 2003

(54) COMPOSITIONS AND METHODS FOR IDENTIFYING AGENTS THAT ALTER MITOCHONDRIAL PERMEABILITY TRANSITION PORES

(76) Inventors: JAMES A. DYKENS, ENCINITAS, CA (US); SCOTT W. MILLER, SAN MARCOS, CA (US); SOUMITRA S. GHOSH, SAN DIEGO, CA (US); ROBERT E. DAVIS, SAN DIEGO, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/161,172

(22) Filed: Sep. 25, 1998

(51) Int. Cl. C12Q 1/68; C12Q 1/00; G01N 33/53

(52) U.S. Cl. 435/6; 435/4; 435/7.1

(57) ABSTRACT

The invention is directed to methods for identifying agents that affect mitochondrial functions and cell death. Such agents are useful for treating diseases associated with mitochondrial dysfunction and to methods of identifying a risk or presence of such diseases. In particular, the invention relates to the loss of mitochondrial membrane potential (ΔΨm) during mitochondrial permeability transition (MPT) and further provides a measurable rate loss function, changes in which are useful, inter alia, for detecting agents that affect one or more mitochondrial functions, for detecting mitochondrial diseases and for studying molecular components of mitochondria that regulate MPT.

### TECHNICAL FIELD

The invention relates to respiratory and metabolic diseases, and in particular to diseases associated with alterations in mitochondrial function.

### BACKGROUND OF THE INVENTION

Mitochondria are the main energy source in cells of higher organisms, and these organelles provide direct and indirect biochemical regulation of a wide array of cellular respiratory, oxidative and metabolic processes. These include electron transport chain (ETC) activity, which drives oxidative phosphorylation to produce metabolic energy in the form of adenosine triphosphate (ATP), and which also underlies a central mitochondrial role in intracellular calcium homeostasis.

Mitochondrial ultrastructural characterization reveals the presence of an outer mitochondrial membrane that serves as an interface between the organelle and the cytosol, a highly folded inner mitochondrial membrane that appears to form attachments to the outer membrane at multiple sites, and an intermembrane space between the two mitochondrial membranes. The subcompartment within the inner mitochondrial membrane is commonly referred to as the mitochondrial matrix. (For a review, see, e.g., Ernster et al., 1981 J. Cell Biol. 91:227s.) The cristae, originally postulated to occur as infoldings of the inner mitochondrial membrane, have recently been characterized using three-dimensional electron tomography as also including tube-like conduits that may form networks, and that can be connected to the inner membrane by open, circular (30 nm diameter) junctions (Perkins et al., 1997 Journal of Structural Biology 119:260). While the outer membrane is freely permeable to ionic and non-ionic solutes having molecular weights less than about ten kilodaltons, the inner mitochondrial membrane exhibits selective and regulated permeability for many small molecules, including certain cations, and is impermeable to large (>˜10 kDa) molecules.

Altered or defective mitochondrial activity, including but not limited to failure at any step of the ETC, may result in the generation of highly reactive free radicals that have the potential of damaging cells and tissues. These free radicals may include reactive oxygen species (ROS) such as superoxide, peroxynitrite and hydroxyl radicals, and potentially other reactive species that may be toxic to cells. For example, oxygen free radical induced lipid peroxidation is a well established pathogenetic mechanism in central nervous system (CNS) injury such as that found in a number of degenerative diseases, and in ischemia (i.e., stroke).

In addition to free radical mediated tissue damage, there are at least two deleterious consequences of exposure to reactive free radicals arising from mitochondrial dysfunction that adversely impact the mitochondria themselves. First, free radical mediated damage may inactivate one or more of the myriad proteins of the ETC. Second, free radical mediated damage may result in catastrophic mitochondrial collapse that has been termed “permeability transition” (PT) or “mitochondrial permeability transition” (MPT). According to generally accepted theories of mitochondrial function, proper ETC respiratory activity requires maintenance of an electrochemical potential (ΔΨm) in the inner mitochondrial membrane by a coupled chemiosmotic mechanism, as described herein. Free radical oxidative activity may dissipate this membrane potential, thereby preventing ATP biosynthesis and halting the production of a vital biochemical energy source. In addition, mitochondrial proteins such as cytochrome c and “apoptosis inducing factor” may leak out of the mitochondria after permeability transition and may induce the genetically programmed cell suicide sequence known as apoptosis or programmed cell death (PCD). Therefore, mere determination of free radical induced damage, such as lipid peroxidation, is not an accurate or early indicator of mitochondrial dysfunction.

Altered mitochondrial function characteristic of the mitochondria associated diseases may also be related to loss of mitochondrial membrane electrochemical potential by mechanisms other than free radical oxidation, and such transition permeability may result from direct or indirect effects of mitochondrial genes, gene products or related downstream mediator molecules and/or extramitochondrial genes, gene products or related downstream mediators, or from other known or unknown causes. Loss of mitochondrial potential therefore may be a critical event in the progression of diseases associated with altered mitochondrial function, including degenerative diseases.

Mitochondrial defects, which may include defects related to the discrete mitochondrial genome that resides in mitochondrial DNA and/or to the extramitochondrial genome, which includes nuclear chromosomal DNA and other extramitochondrial DNA, may contribute significantly to the pathogenesis of diseases associated with altered mitochondrial function. For example, alterations in the structural and/or functional properties of mitochondrial components comprised of subunits encoded directly or indirectly by mitochondrial and/or extramitochondrial DNA, including alterations deriving from genetic and/or environmental factors or alterations derived from cellular compensatory mechanisms, may play a role in the pathogenesis of any disease associated with altered mitochondrial function. A number of degenerative diseases are thought to be caused by, or to be associated with, alterations in mitochondrial function. These include Alzheimer's Disease; diabetes mellitus; Parkinson's Disease; Huntington's disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome. The extensive list of additional diseases associated with altered mitochondrial function continues to expand as aberrant mitochondrial or mitonuclear activities are implicated in particular disease processes.

A hallmark pathology of AD and potentially other diseases associated with altered mitochondrial function is the death, of selected cellular populations in particular affected tissues, which results from apoptosis (also referred to as “programmed cell death”) according to a growing body of evidence. Mitochondrial dysfunction is thought to be critical in the cascade of events leading to apoptosis in various cell types (Kroemer et al., FASEB J. 9:1277-87, 1995), and may be a cause of apoptotic cell death in neurons of the AD brain. Altered mitochondrial physiology may be among the earliest events in PCD (Zamzami et al., J. Exp. Med. 182:367-77, 1995; Zamzami et al., J. Exp. Med. 181:1661-72, 1995) and elevated reactive oxygen species (ROS) levels that result from such altered mitochondrial function may initiate the apoptotic cascade (Ausserer et al., Mol. Cell. Biol. 14:5032-42, 1994). In cell-free systems, mitochondrial, but not nuclear, enriched fractions are capable of inducing nuclear apoptosis (Newmeyer et al., Cell 70:353-64, 1994). Perturbation of mitochondrial respiratory activity leading to altered cellular metabolic states, such as elevated intracellular ROS, may occur in mitochondria associated diseases and may further induce pathogenetic events via apoptotic mechanisms.

Oxidatively stressed mitochondria may release a pre-formed soluble factor that can induce chromosomal condensation, an event preceding apoptosis (Marchetti et al., Cancer Res. 56:2033-38, 1996). In addition, members of the Bc1-2 family of anti-apoptosis gene products are located within the outer mitochondrial membrane (Monaghan et al., J. Histochem. Cytochem. 40:1819-25, 1992) and these proteins appear to protect membranes from oxidative stress (Korsmeyer et al., Biochim. Biophys. Act. 1271:63, 1995). Localization of Bc1-2 to this membrane appears to be indispensable for modulation of apoptosis (Nguyen et al., J. Biol. Chem. 269:16521-24, 1994). Thus, changes in mitochondrial physiology may be important mediators of apoptosis.

Clearly there is a need for compounds and methods that limit or prevent damage to organelles, cells and tissues that may directly or indirectly result from alterations in mitochondrial function including mitochondrial dysfunction, such as mitochondrial permeability transition that is the cause or consequence of oxidative phosphorylation uncoupling and/or intracellular free radical generation. In particular, because mitochondria are essential organelles for a variety of cellular activities including metabolic energy production, aerobic respiration and intracellular calcium regulation, agents and methods that regulate MPT would be especially useful. Such agents and methods may be suitable for the treatment of diseases associated with altered mitochondrial function, including degenerative diseases described above. Existing approaches to the identification of agents useful for such diseases do not include determination of whether such agents alter mitochondrial permeability transition pores or influence mitochondrial structure and/or function. The present invention fulfills these needs and provides other related advantages.

### SUMMARY OF THE INVENTION

The present invention is directed to compositions and methods for treating diseases associated with altered mitochondrial function. More specifically, without wishing to be bound by any theory, according to the present disclosure it may be appreciated, inter alia, that the selective permeability of the inner mitochondrial membrane may depend on the maintenance of membrane potential (ΔΨm), that partial or complete loss of ΔΨm in mitochondrial permeability transition (MPT) may accompany loss of the selective permeability properties of the mitochondrial membrane, that MPT may be quantified as a rate loss function, that the loss of mitochondrial selective permeability may be mediated by a mitochondrial “pore” comprising one or more molecular components that regulate or otherwise affect MPT, that MPT and/or loss of ΔΨm may be indicative of mitochondrial dysfunction and are present in a wide range of diseases associated with altered mitochondrial function, and that sequelae of MPT and loss of ΔΨm may include induction of apoptotic pathways.

These and other aspects of the present invention will become evident upon reference to the following detailed description and attached drawings. In addition, various references are set forth herein which describe in more detail certain aspects of this invention, and are therefore incorporated by reference in their entirety.

### BRIEF DESCRIPTION OF THE DRAWINGS

FIG. 1 shows fluorescent labeling of mitochondria with DASPMI in mixed control (MixCon) cybrid SH-SY5Y cells (FIG. 1A) and loss of DASPMI fluorescence following ionomycin induced MPT (FIG. 1B).

FIG. 2 shows measurement of ionomycin induced MPT with DASPMI as a rate loss function in SH-SY5Y cybrid cells and the effect of cylcosporin on DASPMI loss rate.

FIG. 3 shows measurement of ionomycin induced MPT with DASPMI as a rate loss function in SH-SY5Y cybrid cells and the effect of ruthenium red on DASPMI loss rate.

FIG. 4 shows measurement of atractyloside induced MPT with DASPMI as a rate loss function in control and AD cybrid SH-SY5Y neuroblastoma cells.

FIG. 5 shows measurement of annexin binding to control and AD SH-SY5Y cybrid cells following atractyloside induced MPT.

FIG. 6 shows measurement of caspase-3 activation in control and AD SH-SY5Y cybrid cells following atractyloside induced MPT.

FIG. 7 shows quantification of caspase-3 activation following ionomycin induced MPT in control and AD cybrid SH-SY5Y neuroblastoma cells.

FIG. 8 depicts quantification of cytochrome c release from mitochondria following ionomycin induced MPT in control and AD cybrid SH-SY5Y neuroblastoma cells.

FIG. 9 shows effect of pre-treating control and AD cybrid SH-SY5Y neuroblastoma cells with compound (I) on DASPMI loss rate following ionomycin induced MPT.

FIG. 10 shows morphology of mixed control (MixCon) cybrid SH-SY5Y neuroblastoma cells before ionomycin induced MPT (FIG. 10A), four hours after ionomycin induced MPT (FIG. 10B), and the effect of pre-treatment with compound (I) on cell morphology four hours after ionomycin induced MPT (FIG. 10C).

FIG. 11 depicts the effect of pre-treating control and AD cybrid SH-SY5Y neuroblastoma cells with compound (I) on caspase-3 activation following ionomycin induced MPT.

SYMBOLS AND ABBREVIATIONS

| | |
|---|---|
| Δψm | mitochondrial membrane potential |
| ρ⁰ | essentially completely depleted of mtDNA |
| AD | Alzheimer's Disease |
| AMC | 7-amino-4-methylcoumarin |
| ANOVA | analysis of variance |
| ANT | adenine translocator |
| APOE | apolipoprotein E |
| DASPMI | 2,4-dimethylaminostyryl-N-methylpyridinium |
| DMF | dimethylformamide |
| EAM | energy absorption molecule |
| ETC | electron transport chain |
| FITC | fluorescein isothiocyanate |
| FCCP | carbonyl cyanide p-trifluoro-methoxyphenylhydrazone |
| HBSS | Hank's balanced salt solution |
| JC-1 | 5,5′,6,6′-Tetrachloro-1,1′3,3′-Tetraethylbezimidazol- |
| | carbocyanine Iodide |
| MELAS | Mitochondrial Encephalopthy, Lactic Acidosis and Stroke |
| MERRF | Myoclonic Epilepsy Ragged Red Fiber Syndrome |
| MixCon | mixed control |
| MPT | Mitochondrial Permeability Transition |
| mtDNA | mitochondrial DNA |
| NMDA | N-methyl-D-aspartic acid |
| PBG | 1-phenylbiguanide |
| PCD | Programmed Cell Death |
| PMSF | phenylmethylsulfonate |
| PS | phosphatidylserine |
| PT | Permeability Transition |
| RFU | relative fluorescence unit(s) |
| ROS | reactive oxygen species |
| TMRE | tetramethylrhodamine ethyl ester |
| TMRM | tetramethylrhodamine methyl ester |
| VDAC | voltage dependent anion channel |

### DETAILED DESCRIPTION OF THE INVENTION

The present invention relates in part to the unexpected finding that mitochondrial permeability transition (MPT) can be monitored as a rate loss function for modeling diseases associated with altered mitochondrial function. Such MPT may manifest as a more or less continual state of some or all of a diseased organism's mitochondria, or may be temporally or spatially organized. For example, such MPT can be acute, chronic, intermittent, transient, tissue-specific and/or cell type-specific, or progressively altered over time with regard to one or more of such characteristics.

The invention pertains to the dependence of the selective permeability of the inner mitochondrial membrane on the maintenance along this membrane of an electro chemical potential which, as noted above, relies upon proper functioning of the ETC. By way of background, four of the five multisubunit protein complexes (Complexes I, III, IV and V) that mediate ETC activity are localized to the inner mitochondrial membrane, which is the most protein rich of biological membranes in cells (75% by weight); the remaining ETC complex (Complex II) is situated in the matrix. In at least three distinct chemical reactions known to take place within the ETC, positively-charged protons are moved from the mitochondrial matrix, across the inner membrane, to the intermembrane space. This disequilibrium of charged species creates an electrochemical potential of approximately 220 mV referred to as the “protonmotive force” (PMF), which is often represented by the notation ΔΨ or ΔΨm and represents the sum of the electric potential and the pH differential across the inner mitochondrial membrane (see, e.g., Ernster et al., 1981 J. Cell Biol. 91:227s and references cited therein).

This membrane potential provides the energy contributed to the phosphate bond created when adenosine diphosphate (ADP) is phosphorylated to yield ATP by ETC Complex V, a process that is “coupled” stoichiometrically with transport of a proton into the matrix; ΔΨm is also the driving force for the influx of cytosolic Ca²⁺ into the mitochondrion. Under normal metabolic conditions, the inner membrane is impermeable to proton movement from the intermembrane space into the matrix, leaving ETC Complex V as the sole means whereby protons can return to the matrix. When, however, the integrity of the inner mitochondrial membrane is compromised, as occurs during MPT that may accompany a disease associated with altered mitochondrial function, protons are able to bypass the conduit of Complex V without generating ATP, thereby “uncoupling” respiration because electron transfer and associated proton pumping yields no ATP. Thus, mitochondrial permeability transition involves the opening of a mitochondrial membrane “pore”, a process by which, inter alia, the ETC and ΔΨm are uncoupled, ΔΨm collapses and mitochondrial membranes lose the ability to selectively regulate permeability to solutes both small (e.g., ionic Ca²⁺, Na⁺, K⁺, H⁺) and large (e.g., proteins).

Without wishing to be bound by theory, it is unresolved whether this pore is a physically discrete conduit that is formed in mitochondrial membranes, for example by assembly or aggregation of particular mitochondrial and/or cytosolic proteins and possibly other molecular species, or whether the opening of the “pore” may simply represent a general increase in the porosity of the mitochondrial membrane. In any event, because permeability transition may be potentiated by free radical exposure, MPT may be more likely to occur in the mitochondria of cells from patients having diseases associated with altered mitochondrial function, which cells are chronically exposed to such reactive free radicals. Abnormally elevated cytosolic free calcium concentrations may also induce a dramatic change in mitochondrial inner membrane permeability (see, e.g., Crompton et al., 1993 Basic Res. Cardiol. 88:513; Mehendale et al., 1994 FASEB J. 8:1285).

According to the present invention, useful embodiments may be practiced using mitochondria that exhibit no sign of altered mitochondrial function or any functional defect, preferably under conditions where MPT and/or altered ETC activity may be induced in such mitochondria, for example by artificial means described herein. In certain other preferred embodiments of the invention, it may be desirable to use functionally altered mitochondria or functionally defective mitochondria and to compare the extent of MPT in such mitochondria with that of normally functioning mitochondria, or to compare the extent of MPT in such mitochondria in the presence and absence of an agent that is known or suspected to affect MPT and/or ETC activity, and associated events such as, e.g., cell death. In other preferred embodiments of the invention, the extent of MPT in mitochondria from one cell type or species is compared to the extent of MPT in mitochondria from a second cell type or species in order to screen agents that affect MPT selectively, i.e., in one cell type or species but not the other.

Surprisingly, as provided by the present invention and described below, cells or mitochondria from subjects having a disease associated with altered mitochondrial function, or cybrid cells having mitochondria that exhibit altered function, appear to be more susceptible to stimuli that induce MPT than are cells or mitochondria that exhibit normal function. Thus, according to certain embodiments of the invention, MPT may be monitored in cells or mitochondria from a subject suspected of having a disease associated with altered mitochondrial function, or cybrid cells constructed with mitochondria from such a subject, any of which may be predisposed to MPT by the criteria of altered mitochondrial function, including but not limited to: elevated free radicals, impaired ETC and/or respiratory enzyme activity or disrupted intracellular calcium homeostasis. However, other subcellular events that take place in cells of individuals having diseases associated with altered mitochondrial function, regardless of whether or not free radical reactivity or elevated cytosolic calcium are involved, may also potentiate MPT and should be considered within the scope of the invention. The invention may be practiced with any disease or condition having MPT as a diagnostic, prognostic or clinical parameter.

Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See, e.g., Ernster et al., 1981 J. Cell Biol. 91:227s and references cited; see also Haugland, 1996 Handbook of Fluorescent Probes and Research Chemicals—Sixth Ed., Molecular Probes, Eugene, Oreg., pp. 266-274 and 589-594.). For example, by way of illustration and not limitation, the fluorescent probes 2-,4-dimethylaminostyryl-N-methyl pyridinium (DASPMI) and tetramethylrhodamine esters (such as, e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp. 159-186 and references cited therein; and Molecular Probes On-line Handbook of Fluorescent Probes and Research Chemicals, at http://www.probes.com/handbook/toc.html). Other fluorescent detectable compounds that may be used in the invention include but are not limited to rhodamine 123, rhodamine B hexyl ester, DiOC₆(3), JC-1 [5, 5′, 6,6′-Tetrachloro-1, 1′, 3,3′-Tetraethylbezimidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, Göttingen, Germany; see Sakanoue et al., 1997 J. Biochem. 121:29).

Mitochondrial membrane potential can also be measured by non-fluorescent means, for example by using TTP (tetraphenylphosphonium ion) and a TTP-sensitive electrode (Kamo et al., 1979 J. Membrane Biol. 49:105; Porter and Brand, 1995 Am. J. Physiol. 269:R1213). Those skilled in the art will be able to select appropriate detectable compounds or other appropriate means for measuring ΔΨm. By way of example and not limitation, TMRM is somewhat preferable to TMRE because, following efflux from mitochondria, TMRE yields slightly more residual signal in the endoplasmic reticulicum and cytoplasm than TMRM.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. (See, e.g., Quinn, 1976, The Molecular Biology of Cell Membranes, University Park Press, Baltimore, Md., pp. 200-217 and references cited therein.)

Thus, as provided herein, any experimentally measurable consequence for cells containing mitochondria undergoing MPT may be used, including, for example, measurement of the dissipation of ΔΨ, detection of the loss of mitochondrial intermembrane space proteins such as cytochrome c to the cytoplasm, activation of caspase 3 as a downstream event in the apoptotic signaling cascade (see below), cell death and any other phenotypic, biochemical, biophysical, metabolic, respiratory or other useful parameter the alteration of which may depend on MPT. Agents identified according to the methods of the present invention that are suitable for treatment of a disease associated with altered mitochondrial function may potentiate, impair or alter the frequency and/or occurrence of MPT and/or MPT-related regulatory mechanisms. Particularly preferred are agents that inhibit the appearance of one or more of the above indicators of MPT.

Certain aspects of the present invention as it relates to modeling diseases associated with altered mitochondrial function, involve the relationship between ΔΨ and intracellular calcium homeostasis. Normal alterations of intramitochondrial Ca²⁺ are associated with normal metabolic regulation (Dykens, 1998 in Mitochondria & Free Radicals in Neurodegenerative Diseases, Beal, Howell and Bodis-Wollner, Eds., Wiley-Liss, New York, pp. 29-55; Radi et al., 1998 in Mitochondria & Free Radicals in Neurodegenerative Diseases, Beal, Howell and Bodis-Wollner, Eds., Wiley-Liss, New York, pp. 57-89; Gunter and Pfeiffer, 1991, Am. J. Physiol. 27: C755; Gunter et al., 1994, Am. J. Physiol. 267: 313). For example, fluctuating levels of mitochondrial free Ca²⁺ may be responsible for regulating oxidative metabolism in response to increased ATP utilization, via allosteric regulation of enzymes (reviewed by Crompton and Andreeva, 1993 Basic Res. Cardiol. 88: 513-523;) and the glycerophosphate shuttle (Gunter and Gunter, 1994 J. Bioenerg. Biomembr. 26: 471).

Normal mitochondrial function includes regulation of cytosolic free calcium levels by sequestration of excess Ca²⁺ within the mitochondrial matrix. Depending on cell type, cytosolic Ca²⁺ concentration is typically 50-100 nM. In normally functioning cells, when Ca²⁺ levels reach 200-300 nM, mitochondria begin to accumulate Ca²⁺ as a function of the equilibrium between influx via a Ca²⁺ uniporter in the inner mitochondrial membrane and Ca²⁺ efflux via both Na+dependent and Na⁺ independent calcium carriers. The low affinity of this rapid uniporter mechanism suggests that the primary uniporter function may be to lower cytosolic Ca²⁺ in response to pathological elevation of cytosolic free calcium levels, which may result from ATP depletion and/or abnormal calcium influx across the plasma membrane (Gunter and Gunter, 1994 J. Bioenerg. Biomembr. 26: 471; Gunter et al., 1994 Am. J. Physiol. 267:313). In certain instances, such perturbation of intracellular calcium homeostasis is a feature of diseases associated with altered mitochondrial function, regardless of whether the calcium regulatory dysfunction is causative of, or a consequence of, altered mitochondrial function including MPT.

Mitochondrial calcium levels may also reflect transient low cytosolic concentrations, which, in combination with reduced ATP or other conditions associated with mitochondrial pathology can yield MPT (see Gunter et al., 1998 Biochim. Biophys. Acta 1366:5; Rottenberg and Marbach, 1990, Biochim. Biophys. Acta 1016:87). Generally, in order to practice the present invention on a given set of mitochondria, the extramitochondrial (cytosolic) level of Ca²⁺ is greater than that present within mitochondria. In the case of diseases or disorders, including diseases associated with altered mitochondrial function, mitochondrial or cytosolic calcium levels may vary from the above ranges and may range from, e.g., about 1 nM to about 500 mM, more typically from about 10 nM to about 100 μM and usually from about 20 nM to about 1 μM, where “about” indicates ±10%. A variety of calcium indicators are known in the art including but not limited to fura-2 (McCormack et al., 1989 Biochim. Biophys. Acta 973:420); mag-fura-2; BTC (U.S. Pat. No. 5,501,980); fluo-3, fluo-4 and fluo-5N (U.S. Pat. No. 5,049,673); benzothiaza-1; and benzothiaza-2 (all of which are available from Molecular Probes, Eugene, Oreg.).

Ca²⁺ influx into mitochondria appears to be largely dependent, and may be completely dependent, upon the negative transmembrane electrochemical potential (ΔΨ) established by electron transfer, and such influx fails to occur in the absence of ΔΨ even when an eight-fold Ca²⁺ concentration gradient is imposed (Kapus et al., 1991 FEBS Lett. 282:61). Accordingly, mitochondria may release Ca²⁺ via the uniporter when the membrane potential is dissipated, as occurs with uncouplers like 2,4-dinitrophenol and carbonyl cyanide p-trifluoro-methoxyphenylhydrazone (FCCP).

Thus, according to certain embodiments of the present invention, MPT may be potentiated by influxes of cytosolic free calcium into the mitochondria, as may occur under certain physiological conditions including those encountered by cells of a subject having a disease associated with altered mitochondrial function. As noted above, in certain instances cells exposed to appropriate ionophores or other agents or conditions that directly or indirectly induce calcium fluxes across the plasma membrane into the cytoplasm undergo MPT in response to excessive sequestration of Ca²⁺ in the mitochondrial matrix by mitochondrial calcium regulatory mechanisms. Additionally, a variety of physiologically pertinent agents, including hydroperoxide and free radicals, may synergize with Ca²⁺ to induce MPT (Novgorodov et al., 1991 Biochem. Biophys. Acta 1058: 242; Takeyama et al., 1993 Biochem. J. 294: 719; Guidox et al., 1993 Arch. Biochem. Biophys. 306:139).

Compounds that induce increased cytoplasmic and mitochondrial concentrations of Ca²⁺, including calcium ionophores, are well known to those of ordinary skill in the art, as are methods for measuring intracellular calcium and intramitochondrial calcium (see, e.g., Gunter and Gunter, 1994 J. Bioenerg. Biomembr. 26: 471; Gunter et al., 1998 Biochim. Biophys. Acta 1366:5; McCormack et al., 1989 Biochim. Biophys. Acta 973:420; Orrenius and Nicotera, 1994 J. Neural. Transm. Suppl. 43:1; Leist and Nicotera, 1998 Rev. Physiol. Biochem. Pharmacol. 132:79; and Haugland, 1996, supra) . Accordingly, a person skilled in the art may readily select a suitable ionophore (or another compound that results in increased cytoplasmic and/or mitochondrial concentrations of Ca²⁺) and an appropriate means for detecting intracellular and/or intramitochondrial calcium for use in the present invention, according to the instant disclosure and to well known methods. In addition to ionophores, other compounds that induce increased cytoplasmic and mitochondrial concentrations of Ca²⁺ include but are not limited to thapsigargin, carbachol and amino acid neurotransmitters such as glutamate or N-methyl-D-aspartic acid. As will be appreciated by those familiar with the art, the particular cells that are exposed to a given compound such as glutamate require a receptor therefor, in order for the compound to influence intracellular Ca²⁺ levels. For example, NT-2 teratocarcinoma cells express such glutamate receptors, whereas SH-5YSY neuroblastoma cells do not. Thus, the choice of cell line in which it may be desirable to increase cytoplasmic and mitochondrial calcium levels will determine which compounds are most appropriate.

For example, by way of illustration and not limitation, in certain preferred embodiments of the invention ionomycin (Toeplitz et al., 1979 J. Amer. Chem. Soc. 101:3344) may be used as a calcium ionophore and DASPMI (Haugland, 1996 Handbook of Fluorescent Probes and Research Chemicals—Sixth Ed., Molecular Probes, Eugene, Oreg., pp. 266-274) may be a fluorescent indicator of mitochondrial calcium content. In general, any appropriate compound that results in increased cytoplasmic and/or mitochondrial concentrations of Ca²⁺ and any indicator of mitochondrial membrane potential that permits measuring mitochondrial permeability transition in a biological sample may be used to practice the invention. It is known in the art how to determine suitable concentrations of any such compounds for the uses contemplated herein (see, e.g., Takei and Endo, 1994 Brain Res. 652:65; Hatanaka et al., 1996 Biochem. Biophys. Res. Commun. 227:513).

Because loss of membrane potential causes mitochondria to release Ca²⁺ into the cytosol, the Ca²⁺ load on nearby mitochondria is increased, setting up a chain reaction (Darley-Usmar et al., 1991 Ann. Med. 23:583). Independent of the pathological sequelae of PT collapse, which include increased radical production from uncoupled electron transfer, the ensuing loss of ATP per se may be lethal to aerobically poised cells (Jurkowitz-Alexander et al., 1992 J. Neurochem. 59:344). In addition to a reduced metabolic energy supply, the lack of ATP may exacerbate ΔΨm collapse. Conversely, adding exogenous ATP (but not ADP or AMP) to cells may prevent MPT even when cytosolic Ca²⁺ is present at concentrations that would be sufficient to elicit pore opening in the absence of ATP (Duchen, et al., 1993, Cardiovasc. Res. 27: 1790).

MPT may also be induced by compounds that bind one or more mitochondrial molecular components. Such compounds include, but are not limited to, atractyloside and bongkrekic acid. Methods of determining appropriate amounts of such compounds to induce MPT are known in the art (see, e.g., Beutner et al., 1998 Biochim. Biophys. Acta 1368:7; Obatomi and Bach, 1996 Toxicol. Lett. 89:155; Green and Reed, 1998 Science 281:1309; Kroemer et al., 1998 Annu. Rev. Physiol. 60:619; and references cited therein).

In certain aspects of the invention, an altered mitochondrial state is induced by exposing a biological sample to compositions referred to as “apoptogens” that induce programmed cell death, or “apoptosis”. (Green et al., 1998 Science 281:1309). A variety of apoptogens are known to those familiar with the art and may include by way of illustration and not limitation added to cells D specific receptors required Apoptogen, Tumor necrosis factor, FasL, Glutamate, NMDA, IL-3, Corticosterone, mineralcorticoid or glucocorticoid receptor(s), Herbimycin A, Mancini et al., 1997 J. Cell. Biol. 138:449-469, Paraquat Costantini et al., 1995 Toxicology 99:1-2, Ethylene glycols, http://www.ulaval.ca/vrr/rech/Proj/532866.html, Protein kinase inhibitors, such as, e.g.: Staurosporine, Calphostin C, Caffeic acid phenethyl ester, Chelerythrine chloride, Genistein, 1-(5-isoquinolinesulfonyl)-2-methylpiperazine, N-[2-((p-bromocinnamyl)amino)ethyl]-5-5-isoquinolinesulfonamide, KN-93, Quercitin, d-erythro-sphingosine derivatives, UV radiation, Ionophores such as, e.g.: Ionomycin, valinomycin, MAP kinase inducers such as, e.g.: Anisomycin, Anandamine, Cell cycle blockers such as, e.g.: Aphidicolin, Colcemid, 5-fluorouracil, homoharringtonine, Acetylcholineesterase inhibitors such as, e.g.: berberine, Anti-estrogens such as, e.g.: Tamoxifen, Pro-oxidants, such as, e.g.,: Tert-butyl peroxide, Hydrogen peroxide, Free radicals such as, e.g.: Nitrous oxide, Inorganic metal ions, such as, e.g.: Cadmium, DNA synthesis inhibitors such as, e.g.: Actinomycin D, Intercalators such as, e.g., doxorubicin, Bleomycin sulfate, Hydroxyurea, Methotrexate, Mitomycin C, Camptothecin, daunorubicin, Protein synthesis inhibitors such as, e.g.: cyclohexamide, puromycin, rapamycin, Agents that affect microtubulin formation or stability such as, e.g.: Vinblastine, Vincristine, colchicine, 4-hydroxyphenylretinamide, paclitaxel, (MPT inducers), Bax protein, Jurgenmeier et al., 1998 PNAS 95:4997-5002, Calcium and inorganic phosphate. Kroemer et al., 1998 Ann. Rev. Physiol. 60:619.

As described herein, isolation of a mitochondrial pore component or a mitochondrial molecular species with which an agent identified according to the methods of the invention interacts refers to physical separation of such a complex from its biological source, and may be accomplished by any of a number of well known techniques including but not limited to those described herein, and in the cited references. Without wishing to be bound by theory, a compound that “binds a mitochondrial component” can be any discrete molecule, agent compound, composition of matter or the like that may, but need not, directly bind to a mitochondrial molecular component, and may in the alternative bind indirectly to a mitochondrial molecular component by interacting with one or more additional components that bind to a mitochondrial molecular component. These or other mechanisms by which a compound may bind to and/or associate with a mitochondrial molecular component are within the scope of the claimed methods, so long as isolating a mitochondrial pore component also results in isolation of the mitochondrial molecular species that directly or indirectly binds to the identified agent.

As described herein, the mitochondrial permeability transition “pore” may not be a discrete assembly or multisubunit complex, and the term thus refers instead to any mitochondrial molecular component (including, e.g., a mitochondrial membrane per se) that regulates the inner membrane selective permeability where such regulated function is impaired during MPT. As used herein, mitochondria are comprised of “mitochondrial molecular components”, which may be any protein, polypeptide, peptide, amino acid, or derivative thereof; any lipid, fatty acid or the like, or derivative thereof; any carbohydrate, saccharide or the like or derivative thereof, any nucleic acid, nucleotide, nucleoside, purine, pyrimidine or related molecule, or derivative thereof, or the like; or any other biological molecule that is a constituent of a mitochondrion. “Mitochondrial molecular components” includes but is not limited to “mitochondrial pore components”. A “mitochondrial pore component” is any mitochondrial molecular component that regulates the selective permeability characteristic of mitochondrial membranes as described above, including those responsible for establishing ΔΨm and those that are functionally altered during MPT.

In addition to monitoring Ca²⁺ release, other techniques may be used to follow the progression and extent of MPT and/or MPT-associated events. By way of example and not limitation, other measures of MPT include the exteriorization of plasma membrane phoshatidylserine, release of cytochrome c from mitochondria and induction of specific proteases known as caspases (Green and Reed, 1998 Science 281:1309). Exemplary means of monitoring these processes are described in Examples 5, 7 and 9, respectively, of the present specification.

The present invention provides methods for identifying an agent suitable for treatment of a subject suspected of having a disease associated with altered mitochondrial function by measuring MPT, and thus discloses assays for detecting an agent that influences the effect of any mitochondrial permeability pore component on the permeability properties of the mitochondrial inner membrane. In certain embodiments of the invention, for example, model cell based systems are established in which MPT is induced and detected, as described herein, and further wherein an agent that influences MPT is identified. Accordingly it is understood that the methods of the invention allow for the identification of agents that affect mitochondrial pore activity and may further be used in the identification of known or suspected molecular species that are components of the pore, as well as other molecular components of mitochondria that are responsible for pore properties.

Identification of an agent that affects mitochondrial pore activity according to the present invention provides an agent that may be useful in a pharmaceutical composition. The pharmaceutical composition will include at least one of a pharmaceutically acceptable carrier, diluent or excipient, in addition to one or more agent that affects mitochondrial pore activity and, optionally, other components.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

The pharmaceutical compositions that contain one or more agent that affects mitochondrial pore activity may be in any form which allows for the composition to be administered to a patient. For example, the composition may be in the form of a solid, liquid or gas (aerosol). Typical routes of administration include, without limitation, oral, topical, parenteral (e.g., sublingually or buccally), intrathecal, sublingual, rectal, vaginal, and intranasal. The term parenteral as used herein includes subcutaneous injections, intravenous, intramuscular, intrastemal, intracavemous, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred composition contain, in addition to one or more agent that affects mitochondrial pore activity, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of agent that affects mitochondrial pore activity such that a suitable dosage will be obtained. Typically, this amount is at least 0.01 wt % of an agent that affects mitochondrial pore activity in the composition. When intended for oral administration, this amount may be varied to be between 0.1 and about 70% of the weight of the composition. Preferred oral compositions contain between about 4% and about 50% of agent(s) that affects mitochondrial pore activity. Preferred compositions and preparations are prepared so that a parenteral dosage unit contains between 0.01 to 1% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the agent that affects mitochondrial pore activity compound of from about 0.1 to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol.

In the methods of the invention, the agent(s) that affects mitochondrial pore activity may be administered through use of insert(s), bead(s), timed-release formulation(s), patch(es) or fast-release formulation(s).

It will be evident to those of ordinary skill in the art that the optimal dosage of the agent(s) that affects mitochondrial pore activity may depend on the weight and physical condition of the patient; on the severity and longevity of the physical condition being treated; on the particular form of the active ingredient, the manner of administration and the composition employed. It is to be understood that use of an agent that affects mitochondrial pore activity in a chemotherapy can involve such a compound being bound to an agent, for example, a monoclonal or polyclonal antibody, a protein or a liposome, which assist the delivery of said compound.

Isolation and, optionally, identification and/or characterization of the mitochondrial pore component or components with which an agent that affects mitochondrial pore activity interacts may also be desirable and are within the scope of the invention. Once an agent is shown to alter MPT according to the methods provided herein, those having ordinary skill in the art will be familiar with a variety of approaches that may be routinely employed to isolate the molecular species specifically recognized by such an agent and involved in regulation of MPT, where to “isolate” as used herein refers to separation of such molecular species from the natural biological environment. Techniques for isolating a mitochondrial permeability transition pore component may include any biological and/or biochemical methods useful for separating the complex from its biological source, and subsequent characterization may be performed according to standard biochemical and molecular biology procedures. Those familiar with the art will be able to select an appropriate method depending on the biological starting material and other factors. Such methods may include, but need not be limited to, radiolabeling or otherwise detectably labeling cellular and mitochondrial components in a biological sample, cell fractionation, density sedimentation, differential extraction, salt precipitation, ultrafiltration, gel filtration, ion-exchange chromatography, partition chromatography, hydrophobic chromatography, electrophoresis, affinity techniques or any other suitable separation method that can be adapted for use with the agent with which the mitochondrial pore component interacts. Antibodies to partially purified components may be developed according to methods known in the art and may be used to detect and/or to isolate such components.

Affinity techniques may be particularly useful in the context of the present invention, and may include any method that exploits a specific binding interaction between a mitochondrial pore component and an agent identified according to the invention that interacts with the pore component. For example, because agents that influence MPT can be immobilized on solid phase matrices, an affinity binding technique for isolation of the pore component may be particularly useful. Alternatively, affinity labeling methods for biological molecules, in which a PT-active agent may be modified with a reactive moiety, are well known and can be readily adapted to the interaction between the agent and a pore component, for purposes of introducing into the pore component a detectable and/or recoverable labeling moiety. (See, e.g., Pierce Catalog and Handbook, 1994 Pierce Chemical Company, Rockford, Ill.; Scopes, R. K., Protein Purification: Principles and Practice, 1987, Springer-Verlag, New York; and Hermanson, G. T. et al., Immobilized Affinity Ligand Techniques, 1992, Academic Press, Inc., California; for details regarding techniques for isolating and characterizing biological molecules, including affinity techniques.

Characterization of mitochondrial pore component molecular species, isolated by PT-active agent affinity techniques described above or by other biochemical methods, may be accomplished using physicochemical properties of the pore component such as spectrometric absorbance, molecular size and/or charge, solubility, peptide mapping, sequence analysis and the like. (See, e.g., Scopes, supra.) Additional separation steps for biomolecules may be optionally employed to further separate and identify molecular species that co-purify with mitochondrial pore components. These are well known in the art and may include any separation methodology for the isolation of proteins, lipids, nucleic acids or carbohydrates, typically based on physicochemical properties of the newly identified components of the complex. Examples of such methods include RP-HPLC, ion exchange chromatography, hydrophobic interaction chromatography, hydroxyapatite chromatography, native and/or denaturing one- and two-dimensional electrophoresis, ultrafiltration, capillary electrophoresis, substrate affinity chromatography, immunoaffinity chromatography, partition chromatography or any other useful separation method.

For example, sufficient amounts of a mitochondrial pore protein may be obtained for partial structural characterization by microsequencing. Using the sequence data so generated, any of a variety of well known suitable strategies for further characterizing the pore components may be employed. For example, nucleic acid probes may be synthesized for screening one or more appropriately chosen cDNA libraries to detect, isolate and characterize a cDNA encoding such component(s). Other examples may include use of the partial sequence data in additional screening contexts that are well known in the art for obtaining additional amino acid and/or nucleotide sequence information. See, e.g., Molecular Cloning: A Laboratory Manual, Third Edition, edited by Sambrook, Fritsch & Maniatis, Cold Spring Harbor Laboratory, 1989. Such approaches may further include nucleic acid library screening based on expression of library sequences as polypeptides, such as binding of such polypeptides to PT-active agents identified according to the present invention; or phage display screening approaches or dihybrid screening systems based on protein-protein interactions with known mitochondrial proteins, and the like, any of which may be adapted to screening for PT pore components provided by the present invention using routine methodologies with which those having ordinary skill in the art will be familiar. (See, e.g., Bartel et al., In Cellular Interactions in Development. A Practical Approach, Ed. D. A. Harley, 1993 Oxford University Press, Oxford, United Kingdom, pp. 153-179, and references cited therein.) Preferably extracts of cultured cells, and in particularly preferred embodiments extracts of biological tissues or organs may be sources of novel mitochondrial PT pore proteins. Preferred sources may include blood, brain, fibroblasts, myoblasts, liver cells or other cell types.

Certain mitochondrial molecular components may contribute to the MPT mechanism, including ETC components or other mitochondrial components described herein. For example, adenine nucleotide translocator (ANT) is believed to mediate ATP/proton exchange across the inner mitochondrial membrane, and the ANT inhibitors atractyloside or bongkrekic acid may induce MPT. Three ANT isoforms have been described that differ in their tissue expression patterns. (Wallace et al., 1998 in Mitochondria & Free Radicals in Neurodegenerative Diseases, Beal, Howell and Bodis-Wollner, Eds., Wiley-Liss, New York, pp. 283-307) Other non-limiting examples of mitochondrial or mitochondria associated proteins that appear to contribute to the MPT mechanism include members of the voltage dependent anion channel (VDAC, also known as porin) family of proteins, the mitochondrial calcium uniporter, mitochondria associated hexokinase(s), peripheral benzodiazepine receptor, intermembrane creatine kinases and cyclophilin D. The PT pore may be selectively inhibited by cyclosporin A, which may block MPT by inhibiting cyclophilin D peptidyl-prolyl isomerase activity (Murphy et al., 1998 in Mitochondria & Free Radicals in Neurodegenerative Diseases, Beal, Howell and Bodis-Wollner, Eds., Wiley-Liss, New York, pp. 159-186; White and Reynolds, 1996 J. Neurosci. 16:5688). The role in MPT of these and other mitochondrial molecular components, and factors influencing such components, may be investigated using the invention.

A biological sample containing mitochondria may comprise any tissue or cell preparation in which intact mitochondria capable of maintaining a membrane potential when supplied with one or more oxidizable substrates such as glucose, malate or galactose are or are thought to be present. By “capable of maintaining a potential” it is meant that such mitochondria have a membrane potential that is sufficient to permit the accumulation of the detectable compound (e.g., DASPMI, TMRM, etc.) used in the particular instance. A biological sample may, for example, be derived from a normal (i.e., healthy) individual or from an individual having a disease associated with altered mitochondrial function. Biological samples may be provided by obtaining a blood sample, biopsy specimen, tissue explant, organ culture or any other tissue or cell preparation from a subject or a biological source. The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines, differentiated or differentiable cell lines, transformed cell lines and the like. In particularly preferred embodiments, the subject or biological source is a cybrid cell line produced as known in the art and described herein using ρ⁰ cells or mitochondrial DNA depleted cells that are repopulated with mitochondria from a human or non-human animal subject of interest. (See, e.g., WO 95/26973.) In certain preferred embodiments of the invention, the subject or biological source may have or be at risk for having a disease associated with altered mitochondrial function, and in certain preferred embodiments of the invention the subject or biological source may be known to be free of a risk or presence of such as disease.

In certain other preferred embodiments where it is desirable to determine whether or not a subject or biological source falls within clinical parameters indicative of Alzheimer's disease (AD), signs and symptoms of AD that are accepted by those skilled in the art may be used to so designate a subject or biological source, for example clinical signs referred to in McKhann et al. (Neurology 34:939, 1984, National Institute of Neurology, Communicative Disorders and Stroke and Alzheimer's Disease and Related Disorders Association Criteria of Probable AD, NINCDS-ADRDA) and references cited therein, or other means known in the art for diagnosing AD.

In certain aspects of the invention, biological samples containing mitochondria may be obtained from a subject or biological source before and after contacting the biological sample with a candidate agent, for example to identify a candidate agent capable of effecting a change in mitochondrial inner membrane permeability, as defined above, relative to the mitochondrial inner membrane permeability before exposure of the subject or biological source to the agent.

In a preferred embodiment of the invention, the biological sample containing mitochondria may comprise a crude buffy coat fraction of whole blood, which is known in the art to comprise further a particulate fraction of whole blood enriched in white blood cells and platelets and substantially depleted of erythrocytes. Those familiar with the art will know how to prepare such a buffy coat fraction, which may be prepared by differential density sedimentation of blood components under defined conditions, including the use of density dependent separation media, or by other methods.

According to certain embodiments of the invention, the particular cell type or tissue type from which a biological sample is obtained may influence qualitative or quantitative aspects of the mitochondrial permeability measured therein relative to mitochondrial permeability in distinct cell or tissue types from a common biological source. As described above, some diseases associated with altered mitochondrial function may manifest themselves in particular cell or tissue types. For example, AD is primarily a neurodegenerative disease that particularly effects changes in the central nervous system (CNS). It is therefore within the invention to quantify mitochondrial permeability in biological samples from different cell or tissue types as may render the advantages of the invention most useful for a particular disease associated with altered mitochondrial function, and the relevant cell or tissue types will be known to those familiar with such diseases.

Within the present invention, it is also useful to construct a model system for diagnostic tests and for screening candidate therapeutic agents in which the nuclear genetic background may be held constant while the mitochondrial genome is modified. It is known in the art to deplete mitochondrial DNA from cultured cells to produce ρ⁰ cells, thereby preventing expression and replication of mitochondrial genes and inactivating mitochondrial function. See, for example, International PCT Publication Number WO 95/26973, which is hereby incorporated by reference in its entirety, and references cited therein.

The term “ρ⁰ cells” refers to cells essentially completely depleted of mtDNA, and therefore have no functional mitochondrial respiration/electron transport activity. Such absence of mitochondrial respiration may be established by demonstrating a lack of oxygen consumption by intact cells in the absence of glucose, and/or by demonstrating a lack of catalytic activity of electron transport chain enzyme complexes having subunits encoded by mtDNA, using methods well known in the art. (See, e.g., Miller et al., J. Neurochem. 67:1897-1907, 1996.) That cells have become ρ⁰ cells may be further established by demonstrating that no mtDNA sequences are detectable within the cells. For example, using standard techniques well known to those familiar with the art, cellular mtDNA content may be measured using slot blot analysis of 1 μg total cellular DNA probed with a mtDNA-specific oligonucleotide probe radiolabeled with, e.g., ³²P to a specific activity ≧900 Ci/gm. Under these conditions ρ⁰ cells yield no detectable hybridizing probe signal. Alternatively, any other method known in the art for detecting the presence of mtDNA in a sample may be used that provides comparable sensitivity.

“Mitochondrial DNA depleted” cells (“mtDNA depleted cells”) are cells substantially but not completely depleted of functional mitochondria and/or mitochondrial DNA, by any method useful for this purpose. MtDNA depleted cells are preferably at least about 80% depleted of mtDNA as measured using the slot blot assay described above for the determination of the presence of ρ⁰ cells, and more preferably at least about 90% depleted of mtDNA. Most preferably, mtDNA depleted cells are depleted of greater than about 95% of their mtDNA, wherein “about” indicates ±5% in each instance.

It is further known in the art to repopulate ρ⁰ cells with mitochondria derived from foreign cells in order to assess the contribution of the donor mitochondrial genotype to the respiratory phenotype of the recipient cells. Such cytoplasmic hybrid cells, containing genomic and mitochondrial DNAs of differing biological origins, are known as cybrids. Mitochondria to be transferred to construct cybrids or other model systems in accordance with the present invention may be isolated from virtually any normal or diseased tissue or cell source, including subjects or biological sources known to have or be at risk for having a disease associated with altered mitochondrial function and subjects or biological sources known to be free of such a disease. Cell cultures of all types may potentially be used, as may cells from any tissue. However, fibroblasts, brain tissue, myoblasts and platelets are preferred sources of donor mitochondria. Platelets are the most preferred, in part because of their ready abundance, and their lack of nuclear DNA. This preference is not meant to constitute a limitation on the range of cell types that may be used as donor sources.

For example, platelets may be isolated by an adaptation of the method of Chomyn (Am. J. Hum. Genet. 54:966-974, 1994). However, it is not necessary that this particular method be used; other methods are easily substituted by those skilled in the art. For instance, if nucleated cells are used, cell enucleation and isolation of mitochondria isolation can be performed as described by Chomyn et al., Mol. Cell. Biol. 11:2236-2244, 1991. Human tissue from a subject having or being at risk for having a disease associated with altered mitochondrial function, or from a subject known to be free of a risk or presence of such a disease, may be the source of donor mitochondria. In certain embodiments of the invention, human tissue from a plurality of subjects known to be free of a risk or presence of a disease associated with altered mitochondrial function is used as the source of mitochondria to be transferred into ρ⁰ cells or mtDNA depleted cells to produce cybrid cells.

After preparation of mitochondria by isolation of platelets or enucleation of donor cells, the mitochondria may be transplanted into ρ⁰ cells or mtDNA depleted cells using any known technique for introducing an organelle into a recipient cell, including but not limited to polyethylene glycol (PEG) mediated cell membrane fusion, cell membrane permeabilization, cell-cytoplast fusion, virus mediated membrane fusion, liposome mediated fusion, particle mediated cellular uptake, microinjection or other methods known in the art. For example by way of illustration and not limitation, mitochondria donor cells (˜1×10⁷) are suspended in calcium-free Dulbecco's modified Eagle (DME) medium and mixed with ρ⁰ cells (˜0.5×10⁶) in a total volume of 2 ml for 5 minutes at room temperature. The cell mixture is pelleted by centrifugation and resuspended in 150 μl PEG (PEG 1000, J. T. Baker, Inc., 50% w/v in DME). After 1.5 minutes, the cell suspension is diluted with normal ρ⁰ cell medium containing pyruvate, uridine and glucose, and maintained in tissue culture plates. Medium is replenished daily, and after one week medium lacking pyruvate and uridine is used to inhibit growth of unfused ρ⁰ cells. These or other methods known in the art may be employed to produce cytoplasmic hybrid, or “cybrid”, cell lines. Such cybrids are used according to the present invention as biological samples containing mitochondria, as described herein.

As a non-limiting example, cybrid model systems may be useful for screening candidate agents for treatment of a disease associated with altered mitochondrial function, or for diagnosing a patient suspected of having or being at risk for a disease associated with altered mitochondrial function. According to this example, the patient's mitochondria are used to construct cybrid cells as described above. These cybrid cells may then be propagated in vitro and used to provide a biological sample for the determination of mitochondrial permeability, which can be compared to mitochondrial permeability in a control cybrid cell line constructed with mitochondria from a subject known to be free of disease, or in particularly preferred embodiments, from a plurality of such subjects, as described above. Where it is desirable to compare the influence upon mitochondrial permeability, including the influence upon spontaneous or artificially induced MPT, of mitochondria from different sources, both cybrid cell lines may be constructed from the same ρ⁰ cell line to provide a constant background environment. These and similar uses of model systems according to the invention for screening candidate agents for treatment of, or for determining the risk for or presence of a disease associated with, altered mitochondrial function will be appreciated by those familiar with the art and are within the scope and spirit of the invention.

In addition, although the present invention is directed primarily towards model systems for diseases in which the mitochondria have metabolic alterations, it is not so limited. Conceivably there are disorders wherein mitochondria contain structural or morphological defects or anomalies, and the model systems of the present invention are of value, for example, to find drugs that can address that particular aspect of the disease. Also, there are certain individuals that have or are suspected of having extraordinarily effective or efficient mitochondrial function, and the model systems of the present invention may be of value in studying such mitochondria. Moreover, it may be desirable to put known normal mitochondria into cell lines having disease characteristics, in order to evaluate the influence of mitochondrial alterations on pathogenesis. All of these and similar uses are within the scope of the present invention, and the use of the phrase “mitochondrial alteration” herein should not be construed to exclude such embodiments.

The present invention provides compositions and methods that are useful in pharmacogenomics, for the classification and/or stratification of a subject or a patient population, for instance correlation of one or more traits in a subject with indicators of the responsiveness to, or efficacy of, a particular therapeutic treatment. In one aspect of the invention, measurement of mitochondrial permeability in a biological sample from a subject is combined with identification of the subject's apolipoprotein E (APOE) genotype to determine the risk for, or presence of, Alzheimer's disease (AD) in the subject. The apolipoprotein E type 4 allele (APOE-ε4) allele is a genetic susceptibility factor for sporadic AD and confers a two fold risk for AD (Corder et al., Science 261:921, 1993; see also “National Institute on Aging/Alzheimer's Association Working Group Consensus Statement,” Lancet 347:1091, 1996.). Accordingly, in a preferred embodiment of the invention, the method for determining the risk for or presence of AD in a subject by comparing mitochondrial permeability values will further comprise determining the APOE genotype of the subject suspected of being at risk for AD. By using the combination of the methods for determining mitochondrial permeability as disclosed herein, and methods known in the art for determining APOE genotype, an enhanced ability to detect the relative risk for AD is provided by the instant invention along with other related advantages. Similarly, where APOE genotype and risk for AD are correlated, the present invention provides advantageous methods for identifying agents suitable for treating AD where such agents affect mitochondrial permeability in a biological source.

As described herein, determination of mitochondrial permeability may be used to stratify an AD patient population. Accordingly, in another preferred embodiment of the invention, determination of mitochondrial permeability in a biological sample from an AD subject may provide a useful correlative indicator for that subject. An AD subject so classified on the basis of mitochondrial permeability may then be monitored using AD clinical parameters referred to above, such that correlation between mitochondrial permeability and any particular clinical score used to evaluate AD may be monitored. For example, stratification of an AD patient population according to mitochondrial permeability may provide a useful marker with which to correlate the efficacy of any candidate therapeutic agent being used in AD subjects. In a further preferred embodiment of this aspect of the invention, determination of mitochondrial permeability in concert with determination of an AD subject's APOE genotype may also be useful. These and related advantages will be appreciated by those familiar with the art.

The suitability of a compound for treatment of a subject having a disease associated with altered mitochondrial function may be determined by various assay methods. Such compounds are active in one or more of the following assays for measuring mitochondrial permeability transition, or in any other assay known in the art that directly or indirectly measures induction of MPT, MPT itself or any downstream sequelae of MPT, or that may be useful for identifying mitochondrial permeability pore components (i.e., molecules that regulate MPT). Accordingly, it is also an aspect of the invention to provide compositions and methods for treating a disease associated with altered mitochondrial function by administering a composition that regulates MPT. In preferred embodiments of the invention, identification of agents to be formulated into such compositions may be according to the following assay methods.

### A. Assay for Mitochondrial Permeability Transition (MPT) Using 2-,4-Dimethylaminostyryl-N-Methylpyridinium (DASPMI)

According to this assay, one may determine the ability of a mitochondria protecting agent of the invention to inhibit the loss of mitochondrial membrane potential that accompanies mitochondrial dysfunction. As noted above, maintenance of a mitochondrial membrane potential (ΔΨm) may be compromised as a consequence of mitochondrial dysfunction. This loss of membrane potential, or mitochondrial permeability transition (MPT), can be quantitatively measured using the mitochondria-selective fluorescent probe 2-,4-dimethylaminostyryl-N-methylpyridinium (DASPMI).

Upon introduction into cell cultures, DASPMI accumulates in mitochondria in a manner that is dependent on, and proportional to, mitochondrial membrane potential. If mitochondrial function is disrupted in such a manner as to compromise membrane potential, the fluorescent indicator compound leaks out of the membrane bounded organelle with a concomitant loss of detectable fluorescence. Fluorimetric measurement of the rate of decay of mitochondria associated DASPMI fluorescence provides a quantitative measure of loss of membrane potential, or MPT. Because mitochondrial dysfunction may be the result of multiple factors that directly or indirectly induce MPT as described above (e.g., ROS, calcium flux), agents that retard the rate of loss of DASPMI fluorescence may be effective agents for treating diseases associated with altered mitochondrial function, according to the methods of the instant invention.

### B. Assay of Apoptosis in Cells Treated with Mitochondria Protecting Agents

As noted above, mitochondrial dysfunction may be an induction signal for cellular apoptosis. According to this assay, one may determine the ability of a candidate agent to inhibit or delay the onset of apoptosis. Mitochondrial dysfunction may be present in cells known or suspected of being derived from a subject having a disease associated with altered mitochondrial function, or mitochondrial dysfunction may be induced in normal cells by one or more of a variety of physiological and biochemical stimuli, with which those having skill in the art will be familiar.

In one aspect of the apoptosis assay, cells that are suspected of undergoing apoptosis may be examined for morphological, permeability or other changes that are indicative of an apoptotic state. For example by way of illustration and not limitation, apoptosis in many cell types may cause altered morphological appearance such as plasma membrane blebbing, cell shape change, loss of substrate adhesion properties or other morphological changes that can be readily detected by those skilled in the art using light microscopy. As another example, cells undergoing apoptosis may exhibit fragmentation and disintegration of chromosomes, which may be apparent by microscopy and/or through the use of DNA specific or chromatin specific dyes that are known in the art, including fluorescent dyes. Such cells may also exhibit altered plasma membrane permeability properties as may be readily detected through the use of vital dyes (e.g., propidium iodide, trypan blue) or by the detection of lactate dehydrogenase leakage into the extracellular milieu. These and other means for detecting apoptotic cells by morphologic criteria, altered plasma membrane permeability and related changes will be apparent to those familiar with the art.

In another aspect of an apoptosis assay, translocation of cell membrane phosphatidylserine (PS) from the inner to the outer leaflet of the plasma membrane is detected by measuring outer leaflet binding by the PS-specific protein armexin. (Martin et al., J. Exp. Med. 182:1545, 1995; Fadok et al., J. Immunol. 148:2207, 1992.) In another aspect of the apoptosis assay, induction of specific protease activity in a family of apoptosis-activated proteases known as the caspases is measured, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art (see, e.g., Ellerby et al., 1997 J. Neurosci. 17:6165). The synthetic peptide Z-Tyr-Val-Ala-Asp-AFC (SEQ ID NO:1; Example 6), wherein “Z” indicates a benzoyl carbonyl moiety and AFC indicates 7-amino-4-trifluoromethylcoumarin (Kluck et al., 1997 Science 275:1132; Nicholson et al., 1995 Nature 376:37), is one such substrate. Other substrates include nuclear proteins such as U1-70 kDa and DNA-PKcs (Rosen and Casciola-Rosen, 1997 J. Cell Biochem. 64:50; Cohen, 1997 Biochem. J. 326:1).

As described above, the mitochondrial inner membrane may exhibit highly selective and regulated permeability for many small molecules, including certain cations, but is impermeable to large (>˜10 kDa) molecules. (See, e.g., Quinn, 1976 The Molecular Biology of Cell Membranes, University Park Press, Baltimore, Md.). Thus, in another aspect of the apoptosis assay, detection of the mitochondrial protein cytochrome c that has leaked out of mitochondria in apoptotic cells may provide an apoptosis indicator that can be readily determined. (Liu et al., Cell 86:147, 1996) Such detection of cytochrome c may be performed spectrophotometrically, immunochemically or by other well established methods for determining the presence of a specific protein.

Release of cytochrome c from cells challenged with apoptotic stimuli (e.g., ionomycin, a well known calcium ionophore) can be followed by a variety of immunological methods. Matrix-assisted laser desorption ionization time-of-flight (MALDI-TOF) mass spectrometry coupled with affinity capture is particularly suitable for such analysis since apo-cytochrome c and holo-cytochrome c can be distinguished on the basis of their unique molecular weights. For example, the Surface-Enhanced Laser Desorption/Ionization (SELDI™) system (Ciphergen, Palo Alto, Calif.) may be utilized to follow the inhibition by mitochondria protecting agents of cytochrome c release from mitochondria in ionomycin treated cells. In this approach, a cytochrome c specific antibody immobilized on a solid support is used to capture released cytochrome c present in a soluble cell extract. The captured protein is then encased in a matrix of an energy absorption molecule (EAM) and is desorbed from the solid support surface using pulsed laser excitation. The molecular mass of the protein is determined by its time of flight to the detector of the SELDI™ mass spectrometer.

The person of ordinary skill in the art will readily appreciate that there may be other suitable techniques for quantifying apoptosis, and such techniques for purposes of determining the effects of mitochondria protecting agents on the induction and kinetics of apoptosis are within the scope of the assays disclosed here.

### C. Assay of Electron Transport Chain (ETC) Activity in Isolated Mitochondria

As described above, mitochondria associated diseases may be characterized by impaired mitochondrial respiratory activity that may be the direct or indirect consequence of elevated levels of reactive free radicals such as ROS, of elevated cytosolic free calcium concentrations or other stimuli. Accordingly, a suitable agent for use in the treatment of a disease associated with altered mitochondrial function may restore or prevent further deterioration of ETC activity in mitochondria of individuals having mitochondria associated diseases. Assay methods for monitoring the enzymatic activities of mitochondrial ETC Complexes I, II, III, IV and ATP synthetase, and for monitoring oxygen consumption by mitochondria, are well known in the art. (See, e.g., Parker et al., Neurology 44:1090-96, 1994; Miller et al., J. Neurochem. 67:1897, 1996.) It is within the scope of the methods provided by the instant invention to identify a suitable agent using such assays of mitochondrial function, given the relationship between mitochondrial membrane potential and ETC activity as described above. Further, mitochondrial function may be monitored by measuring the oxidation state of mitochondrial cytochrome c at 540 nm. Also as described above, oxidative damage that may arise in mitochondria associated diseases may include damage to mitochondrial components such that the oxidation state of cytochrome c, by itself or in concert with other parameters of mitochondrial function including but not limited to mitochondrial oxygen consumption, may be an indicator of reactive free radical damage to mitochondrial components. Accordingly, the invention provides various assays designed to test the inhibition of such oxidative damage by candidate agents that may influence mitochondrial membrane permeability. The various forms such assays may take will be appreciated by those familiar with the art, and are not intended to be limited by the disclosures herein, including in the Examples.

For example by way of illustration and not limitation, Complex IV activity may be determined using commercially available cytochrome c that is fully reduced via exposure to excess ascorbate. Cytochrome c oxidation may then be monitored spectrophotometrically at 540 nm using a stirred cuvette in which the ambient oxygen above the buffer is replaced with argon. Oxygen reduction in the cuvette may be concurrently monitored using a micro oxygen electrode with which those skilled in the art will be familiar, where such an electrode may be inserted into the cuvette in a manner that preserves the argon atmosphere of the sample, for example through a sealed rubber stopper. The reaction may be initiated by addition of a cell homogenate or, preferably a preparation of isolated mitochondria, via injection through the rubber stopper. In the assay described here, for example, a defect in complex IV activity may be correlated with an enzyme recognition site. This assay, or others based on similar principles, may permit correlation of mitochondrial respiratory activity with mitochondria membrane permeability, which may be determined according to other assays described herein.

Another embodiment of the invention involves its use identifying agents that increase the degree or enhance the rate of apoptosis in hyperproliferative cells present in diseases and disorders such as cancer and psoriasis (note that, for the purposes of the disclosure, the term “hyperproliferative disease or disorder” specifically excludes pregnancy). Because oncogenic changes render certain tumors more susceptible to apoptosis (Evan and Liftlewood, 1998 Science 281:1317), such agents are expected to be useful for treating such hyperproliferative diseases or disorders. In a related embodiment, a biological sample from a patient having or suspected of having a hyperproliferative disease or disorder are evaluated for their susceptibility to such agents using the methods of the invention. Cybrid cells are a preferred biological sample in this embodiment.

A further embodiment of the invention involves its use in identifying agents that alter mitochondrial function and/or selectively affect MPT in mitochondria and/or cell death in a species-specific manner. By “species-specific manner” it iw meant that such agents affect MPT or cell death in a first organism belonging to one species but not in a second organism belonging to another species. This embodiment of the invention is used in a variety of methods.

For example, this embodiment of the invention to identify agents that selectively induce MPT and/or apoptosis in biological samples comprising cells or mitochondria derived from different species, e.g., in trypanasomes (Ashkenazi and Dixit, 1998 Science 281:1305), and other eukaryotic pathogens and parasites, including but not limited to insects, but which do not induce MPT and/or apoptosis in their mammalian hosts. Such agents are expected to be useful for the prophylactic or therapeutic management of such pathogens and parasites.

As another example, this embodiment of the invention is used to identify agents that selectively induce MPT and/or apoptosis in biological samples comprising cells or mitochondria derived from undesirable plants (e.g., weeds) but not in desirable plants (e.g., crops), or in undesirable insects (in particular, members of the family Lepidoptera and other crop-damaging insects) but not in desirable insects (e.g., bees) or desirable plants. Such agents are expected to be useful for the management and control of such undesirable plants and insects. Cultured insect cells, including for example, the Sf9 and Sf21 cell lines derived from Spodoptera frugiperda, and the HIGH FIVE™ cell line from Trichopolusia ni (these three cell lines are available from InVitrogen, Carlsbad, Calif.) may be biological sample in certain such embodiments of the invention.

The following examples are offered by way of illustration, and not by way of limitation.

### EXAMPLES

### Example 1

### Assay for Mitochondrial Permeability Transition Using DASPMI

The fluorescent mitochondria-selective dye 2-,4-dimethylaminostyryl-N-methylpyridinium (DASPMI, Molecular Probes, Inc., Eugene, Oreg.) is dissolved in Hank's balanced salt solution (HBSS; Life Technologies, Rockville, Md.) at 1 mM and diluted to 25 μM in warm HBSS. In 96-well microculture plates, monolayers of cultured human cytoplasmic hybrid (cybrid) cells produced by fusing mitochondrial DNA depleted (ρ⁰) SH-SY5Y cells and mitochondria source platelets (Miller et al., 1996 J. Neurochem. 67:1897-1907) from an individual known or suspected of having a disease associated with altered mitochondrial function, or from a pool (“MixCon”) of platelets provided by several (typically three) normal donors known to be free of disease (“mixed control”), or unmodified SH-SY5Y parental neuroblastoma cells (Biedler et al., 1973 Cancer Res. 33:2643; Biedler et al., 1978 Cancer Res. 38:3751) at or near confluence (i.e., ˜120,000 cells/well), are incubated for 0.5-1.5 hrs in 25 μM DASPMI in a humidified 37 C/5% CO₂ incubator to permit mitochondrial uptake of the fluorescent dye. Culture supernatants are then removed and various concentrations of candidate agents diluted into HBSS from DMF stocks, or vehicle controls, are added. Candidate agents that may affect mitochondria permeability transition (MPT) are introduced to cells from about 5 to about 20 minutes before the exposing the cells to ionomycin (described below), wherein “about” indicates ±10%.

Fluorescence of each microculture in the 96-well plate is quantified immediately using a Molecular Devices fmax fluorimetric plate reader (Molecular Devices Corp., Sunnyvale, Calif.; excitation wavelength=485 nm; emission wavelength=590 nm) and zero-time (t₀) fluorescence is recorded. Thereafter, induction of mitochondrial membrane potential collapse is initiated by the addition of ionomycin (Calbiochem, San Diego, Calif.). Ionomycin stock solutions of various concentrations from 0.1-40 μM are prepared in warm Hank's balanced salt solution (HBSS) and diluted for addition to cells to achieve a final concentration of 0.05-20 μM, with final concentrations of 4-10 μM being preferred. Fluorescence decay of DASPMI-loaded, ionomycin induced cells is monitored as a function of time from 0-500 seconds following addition of ionomycin. The maximum negative slope (V-max) is calculated from a subset of the data using analysis software provided by the fluorimetric plate reader manufacturer. In addition, the initial and final signal intensities are determined and the effects of candidate agents that may affect MPT on the rate of signal decay are quantified.

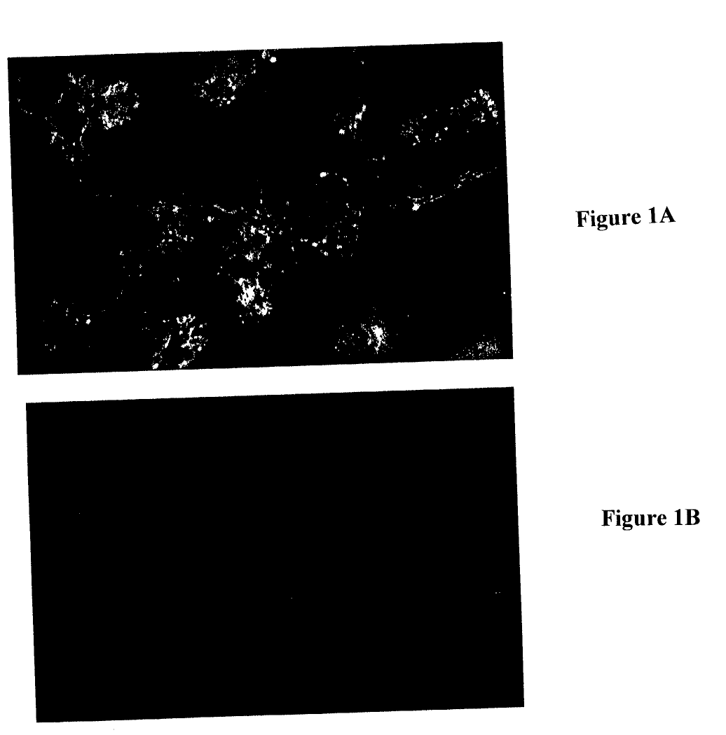

The fluorescence photomicrograph in FIG. 1A shows mitochondrial labeling in mixed control SH-SY5Y neuroblastoma cybrid cells after being exposed to 75 μM DASPMI for one hour in culture, as described above. MPT was then induced in these cells by contacting them with 1 μM ionomycin. FIG. 1B illustrates the collapse of mitochondrial membrane potential and concomitant loss of mitochondria-associated DASPMI fluorescence ten minutes after exposure to ionomycin.

### Example 2

### Inhibition of Ionomycin Induced MPT by Cyclosporin Using the DASPMI Assay

The method described in Example 1 was employed to monitor MPT induced by the calcium ionophore ionomycin and inhibition thereof by cyclosporin. Three cybrid cell lines were produced by fusing ρ⁰ SH-SY5Y neuroblastoma cells with pooled control platelets from cognitively normal, age-matched control donors or platelets from either of two patients diagnosed as having Alzheimer's disease (AD), as described above. Mitochondrial membrane potential-dependent labeling of mitochondria with DASPMI, fluorimetric detection of DASPMI and induction of MPT with ionomycin (5 μM) were as described in Example 1. Cultures of each cybrid cell line were incubated in unmodified media or in media containing 10 μM cyclosporin (CalBiochem-Novabiochem Corp., San Diego, Calif.) diluted from a 22 mM stock solution in DMSO for 10 minutes prior to MPT induction with ionomycin. Fluorescence detection and monitoring of fluorescence decay as a rate loss function were as described in Example 1.

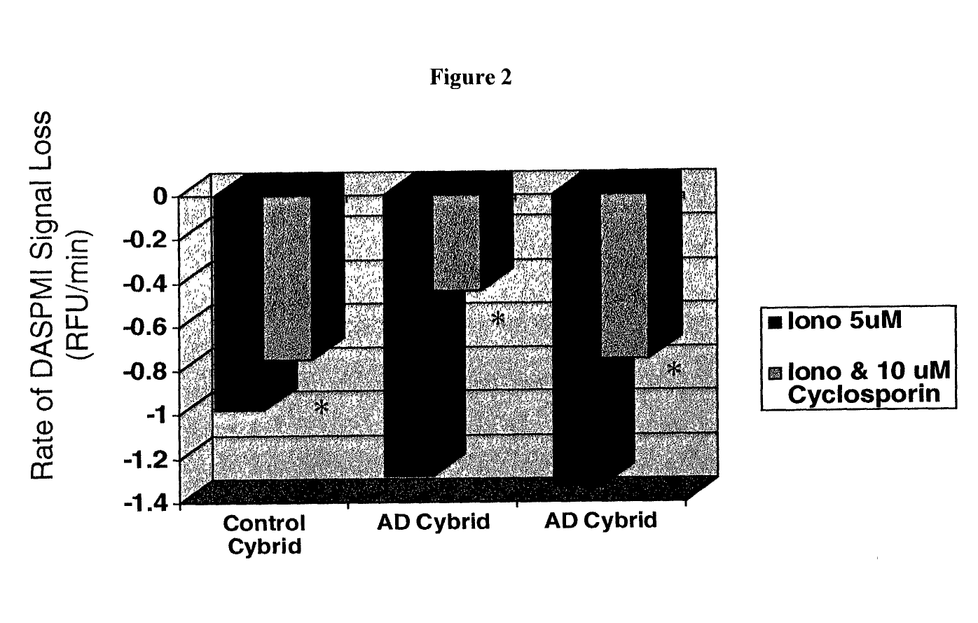

As shown in FIG. 2, DASPMI fluorescence loss rate as an indicator of mitochondrial membrane potential is significantly greater (p<0.0001, as determined by ANOVA (Analysis Of Variance) using MICROSOFT™ Excel) in the two AD cybrid cell lines compared to the control cybrid cell line. As also shown in FIG. 2, treatment of a given cybrid cell line with cyclosporin prior to induction of MPT significantly retards the DASPMI fluorescence loss rate.

### Example 3

### Inhibition of Ionomycin Induced MPT by Ruthenium Red Using the DASPMI Assay

Two cybrid cell lines were produced by fusing ρ⁰ SH-SY5Y neuroblastoma cells with pooled control platelets from cognitively normal, age-matched control donors or platelets from a patient diagnosed as having Alzheimer's disease (AD), as described above. Mitochondrial membrane potential-dependent labeling of mitochondria with DASPMI, fluorimetric detection of DASPMI and induction of MPT with ionomycin (5 μM) were as described in Example 1. Cultures of each cybrid cell line were incubated in unmodified media or in media containing 10 nM ruthenium red (Sigma Chemical Co., St. Louis, Mo.) diluted from a 1 mM stock for 10 minutes prior to MPT induction with ionomycin. Fluorescence detection and monitoring of fluorescence decay as a rate loss function were as described in Example 1.

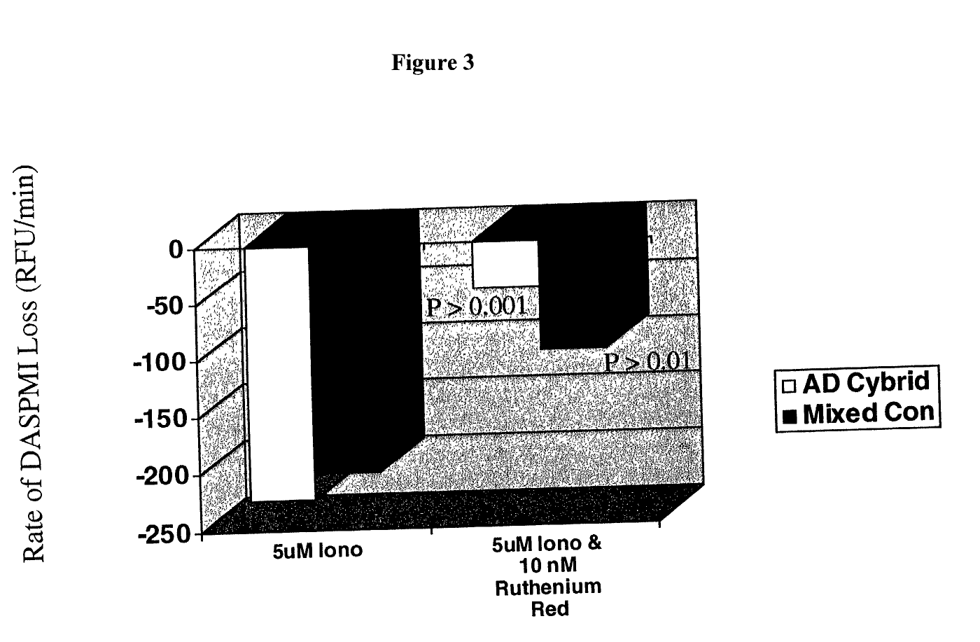

As shown in FIG. 3, DASPMI fluorescence loss rate as an indicator of mitochondrial membrane potential is significantly greater in cybrid cells that have not been treated with ruthenium red than in the cybrid cells that were pretreated with ruthenium red, which inhibits mitochondrial uptake of cytosolic free calcium (Masuoka et al., 1990 Biochem. Biophys. Res. Commun. 169:315).

### Example 4

### Atractyloside Induced MPT Using the DASPMI Assay is Accelerated in AD Cybrids Relative to Control Cybrids

Two cybrid cell lines were produced by fusing ρ⁰ SH-SY5Y neuroblastoma cells either with pooled control platelets from cognitively normal, age-matched control donors or with platelets from a patient diagnosed as having Alzheimer's disease (AD), as described above. Mitochondrial membrane potential-dependent labeling of mitochondria with DASPMI, fluorimetric detection of DASPMI and induction of MPT were as described in Example 1, except that MPT induction was with 2.5 mM atractyloside (CalBiochem-Novabiochem Corp., San Diego, Calif.) in HBSS instead of with ionomycin. Cultures of the parental SH-SY5Y cell line and each cybrid cell line were monitored beginning immediately upon MPT induction with atractyloside. Fluorescence detection and monitoring of fluorescence decay as a rate loss function were as described in Example 1.

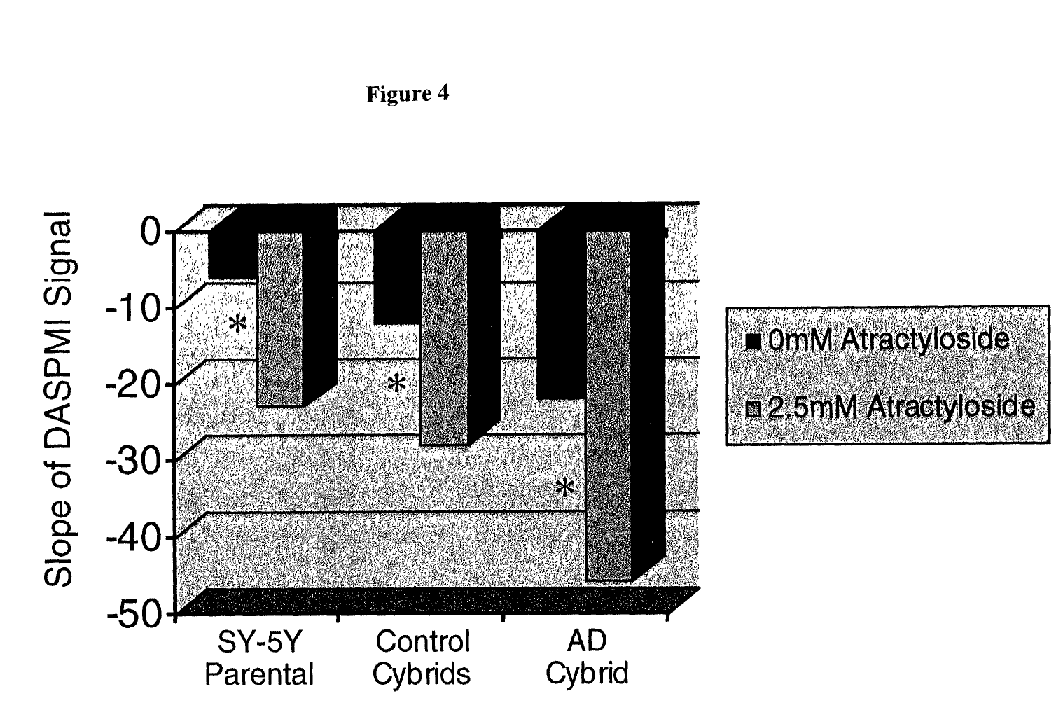

As shown in FIG. 4, following induction of MPT with atractyloside the DASPMI fluorescence loss rate as an indicator of mitochondrial membrane potential is significantly (p<0.01) greater in the AD cybrid cell lines than in the control cybrid cell line or the parental cell line.

### Example 5

### Atractyloside Induced Apoptosis Using the Annexin Assay for Cell Surface Phosphatidylserine is Accelerated in AD Cybrids Relative to Control Cybrids

Preparation of parental SH-SY5Y cells, control cybrid cells and AD cybrid cells and induction of MPT using atractyloside were as described in Example 4. Cells that became apoptotic following MPT were detected by binding of a detectably labeled annexin V derivative (annexin-FITC) to cell surfaces as follows.

Exteriorization of plasma membrane phosphatidylserine (PS) was assessed by adding to the 96 well plate annexin-fluorescein isothiocyanate conjugate (annexin-FITC, Oncogene Research Products, Cambridge, Mass.) dissolved in a suitable buffer for binding to cell surfaces at a final concentration of 5 μg/well, according to the manufacturer's recommendations. (Martin et al., J. Exp. Med. 182:1545, 1995) After 15-30 min in a humidified 37° C./ 5% CO₂ incubator, cells were fixed in situ using 2% formalin, washed to remove non-specifically bound FITC and read using a Cytofluor fluorimetric plate reader (model #2350, Millipore Corp., Bedford, Mass.; excitation wavelength=485 nm; emission wavelength=530 nm) to quantify cell surface bound annexin-FITC as a measure of outer leaflet PS, a marker for cells undergoing apoptosis.

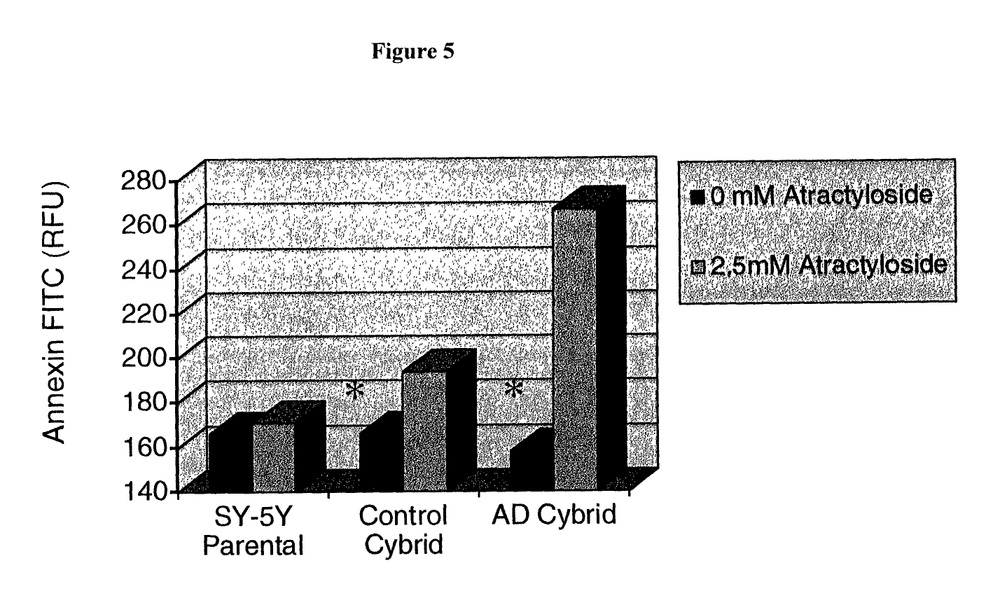

As shown in FIG. 5, following atractyloside induced MPT a significantly (p<0.01) greater proportion of cell surface PS is detectable on AD cybrid cells relative to either control cybrid or parental SH-SY5Y cells, indicative of increased apoptosis in the AD cybrid cell population undergoing MPT.

### Example 6

### lnduced of MPT Induces Apoptosis

In 96-well microculture plates, cultured human cybrid neuroblastoma SH-SY5Y cells constructed using mitochondria from an individual known to have AD, or from a normal control subject, were cultured in HBSS followed by the addition of atractyloside (as described in Example 5) or ionomycin (as described in Example 1). Control cultures, to which neither atractyloside nor ionomycin were added, were prepared in parallel. Membrane potential was monitored from about 15 minutes (which was, in most cases, sufficient for purposes of the assay) to about 45 to 60 minutes, wherein “about” indicates ±10%.

Caspase-3 activity was assessed by diluting the fluorogenic peptide substrate acetyl-Asp-Glu-Val-Asp (SEQ ID NO:2) conjugated to AMC (7-amino-4-methylcoumarin; the synthetic peptide is referred to as DEVD-AMC; CalBiochem-Novabiochem Corp., San Diego, Calif.; see Walker et al., 1994 Cell 78:343, and Thornberry et al., 1992 Nature 356:768) from a DMSO stock solution into culture media to a final concentration of 20 μM for uptake by cells. Substrate cleavage liberating the AMC fluorophore was measured continuously using a Cytofluor fluorimetric plate reader (model #2350, Millipore Corp., Bedford, Mass.; excitation wavelength=435 nm; emission wavelength=460 nm). Data are presented as ΔRFU (relative fluorescence units). Caspase-1 activity (not shown) was measured using the same protocol as that just described for caspase-3, except the caspase-1 specific fluorogenic substrate Z-Tyr-Val-Ala-Asp-AFC (SEQ ID NO:1; Example 6), wherein “Z” indicates a benzoyl carbonyl moiety and AFC indicates 7-amino-4-trifluoromethylcoumarin (CalBiochem-Novabiochem Corp., San Diego, Calif.) is substituted for DEVD-AMC and fluorimetry is conducted using 405 nm excitation and 510 nm emission. Caspase 3 is generally regarded as a mitochondrial-specific caspase, whereas caspase 1 is not; accordingly, DEVD-AMC is one preferred substrate for this embodiment of the invention.

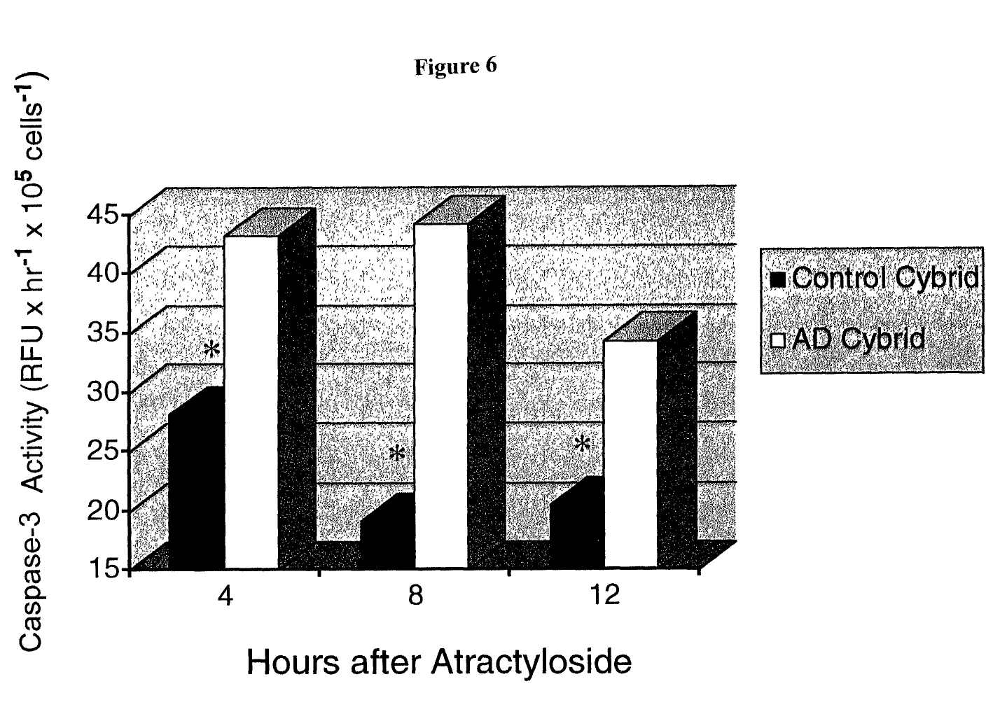

FIG. 6 shows caspase-3 activation, an indicator of apoptosis, following atractyloside induced MPT in control and AD cybrid cells. Significantly (p<0.05, ANOVA as described supra) increased and sustained apoptosis is apparent in cybrid cells constructed with mitochondria from an AD patient, relative to control cybrid cells.

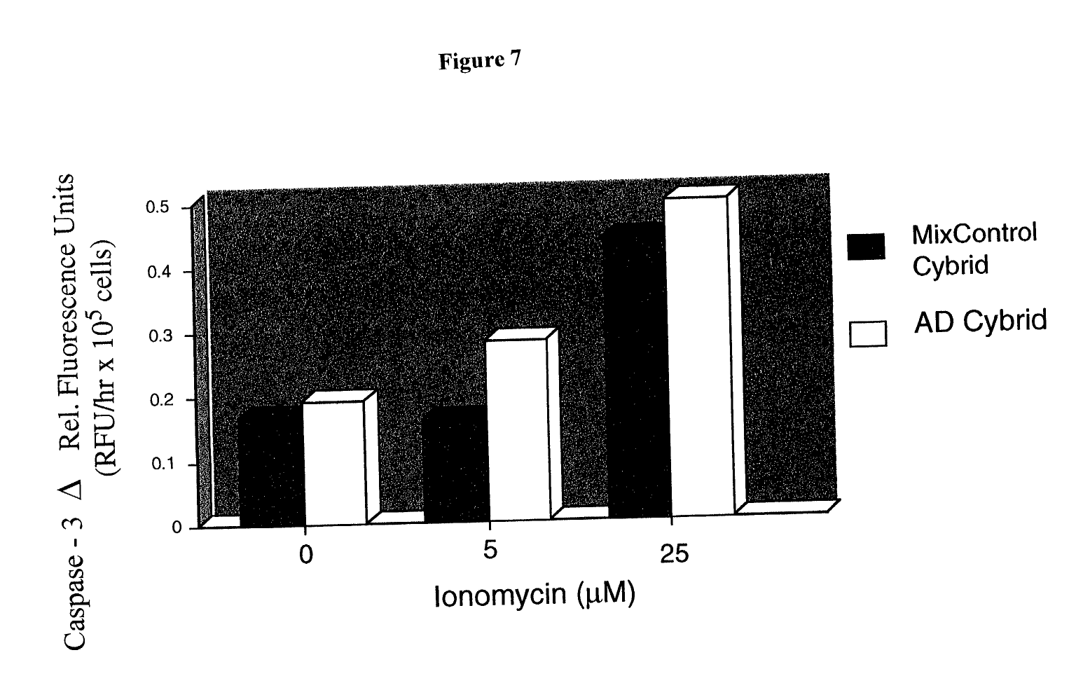

FIG. 7 shows caspase-3 activation following eight hours of ionomycin induced MPT in control and AD cybrid cells. Significantly (p<0.05) increased and sustained apoptosis is apparent in cybrid cells constructed with mitochondria from an AD patient, relative to control cybrid cells.

### Example 7

### Induction of MPT with Ionomycin Induces Apoptosis Detectable by Release of Cytochrome C from Mitochondria

Control cybrid cells (MixCon) produced by fusing ρ⁰ SH-SY5Y neuroblastoma cells with pooled mitochondria source platelets from (typically three) normal subjects, and an AD cybrid cell line produced by fusing ρ⁰ SH-SYSY cells with mitochondria source platelets from an Alzheimer's Disease patient (Miller et al., 1996 J. Neurochem. 67:1897-1907), were grown to complete confluency in 6-well plates (˜3×10⁶ cells/well). Cells were first rinsed with one volume 1×PBS, and then treated with 10 μM ionomycin in DMEM supplemented with 10% FCS, for 1 minute. Cells were then rinsed twice with five volumes of cold 1×PBS containing a cocktail of protease inhibitors (2 μg/ml pepstatin, leupeptin, aprotinin, and 0.1 mM PMSF), and then collected in one ml of cold cytosolic extraction buffer (210 mM mannitol, 70 mM mannitol, 5 mM each of HEPES, EGTA, glutamate and malate, 1 mM MgCl₂, and the protease inhibitor cocktail at the concentrations given above). Homogenization was carried out using 25 strokes with a type B dounce homogenizer on ice. Homogenates were centrifuged at maximum speed (14,000×g) in an Eppendorf (Madison, Wis.) microfuge for five minutes to separate cytosol from intact cells, as well as cell membranes and organelles. The supernatant was collected and an aliquot was saved, along with the pellet, at −80° C. for citrate synthase and protein assays.

Cytochrome c antibody was covalently bound to solid support chips containing a pre-activated surface (PROTEINCHIP™, Ciphergen, Palo Alto, Calif.). The surface area to be treated with antibody was initially hydrated with 1 μl of 50% CH₃CN, and the antibody solution was added before the CH₃CN evaporated. The concentration of the antibody was approximately 1 mg/ml in either NA₃PO₄ or PBS buffer (pH 8.0). The chip was placed in a humid chamber and stored at 4° C. overnight. Prior to addition of the cytosolic extract, residual active sites were blocked by treatment with 1.5 M ethanolamine (pH 8.0) for thirty minutes. The ethanolamine solution was removed and the entire chip was washed in a 15 ml conical tube with 10 ml 0.05% Triton-X 100 in 1×PBS, for 5 minutes with gentle shaking at room temperature. The wash buffer was removed and the chip was sequentially washed, first with 10 ml 0.5 M NaCl in 0.1 M NaOAc (pH 4.5), and then with 0.5 M NaCl in 0.1M Tris (pH 8.0). After removal of the Tris-saline buffer, the chip was rinsed with 1×PBS and was ready for capture of the antigen.

Fresh supernatant samples were spotted onto the Ciphergen ProteinChip containing covalently-linked anti-cytochrome c antibody (Pharmingen, San Diego, Calif.). For optimal antibody-cytochrome c interaction, 100 μl of the supernatant was used and the incubation was carried out overnight with shaking at 4° C. in a Ciphergen bioprocessing unit. The supernatant was then removed and the spots on the chip were washed in the bioprocessing unit three times with 200 μl of 0.1% Triton-X 100 in 1×PBS, and then twice with 200 μl of 3.0 M urea in 1×PBS. The chips were then removed from the bioprocessor and washed with approximately 10 ml of dH₂O. The chips were then dried at room temperature prior to the addition of EAM solution (e.g., sinapinic acid, Ciphergen, Palo Alto, Calif.). A suspension of the EAM was made at a concentration of 25 mg/ml in 50% CH₃CN/H₂O containing 0.5% TFA. The saturated EAM solution was clarified by centrifugation and the supernatant was used for spotting on the ProteinChip surface. Prior to the addition of EAM to the chip, an internal standard of ubiqutin was added to the EAM solution to provide a final concentration of 1 pmol/μl. The quantification of cytochrome c released from mitochondria upon ionomycin treatment was based on normalization to the ubiquitin peak in the mass spectrum and the protein content of the cytosolic extracts. Citrate synthase activity of cytosolic extracts was measured to rule out the possibility of mitochondrial lysis during the sample preparation procedure.

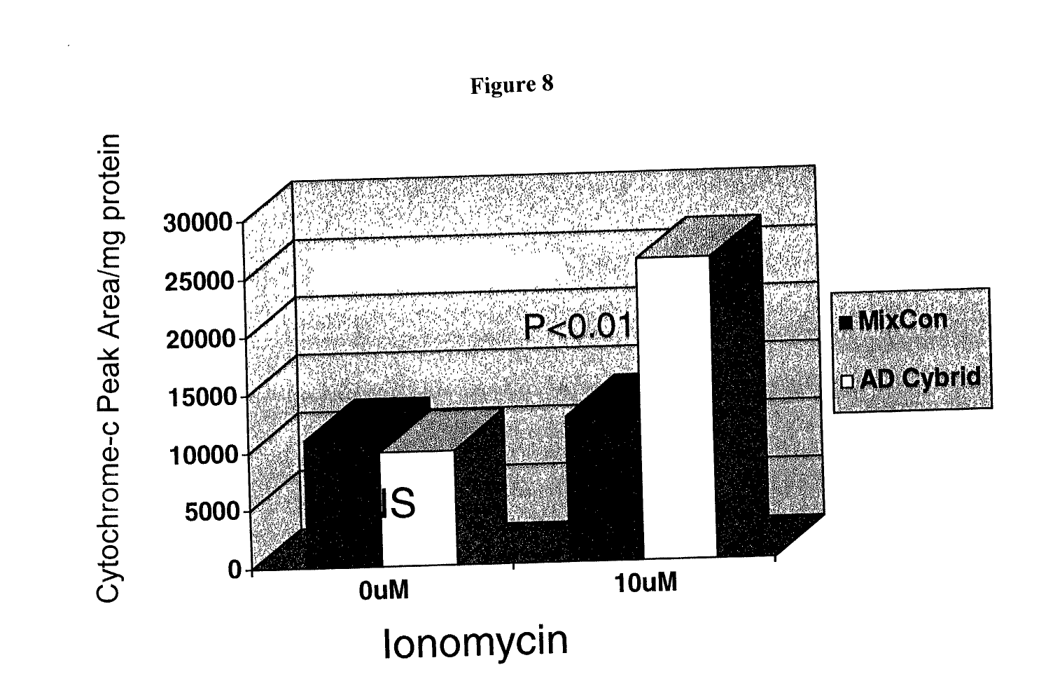

Representative data depicting cytochrome c release in cells undergoing ionomycin induced apoptosis are presented in FIG. 8. As shown in FIG. 8, significantly (p<0.01, ANOVA as described supra) greater quantities of cytochrome c were released from the mitochondria of AD cybrids undergoing ionomycin induced MPT than were released by the mitochondria of control cybrid cells.

### Example 8

### Identification of an Agent that Regulates MPT by Monitoring DASPMI Loss Rate Following Ionomycin Induced MPT

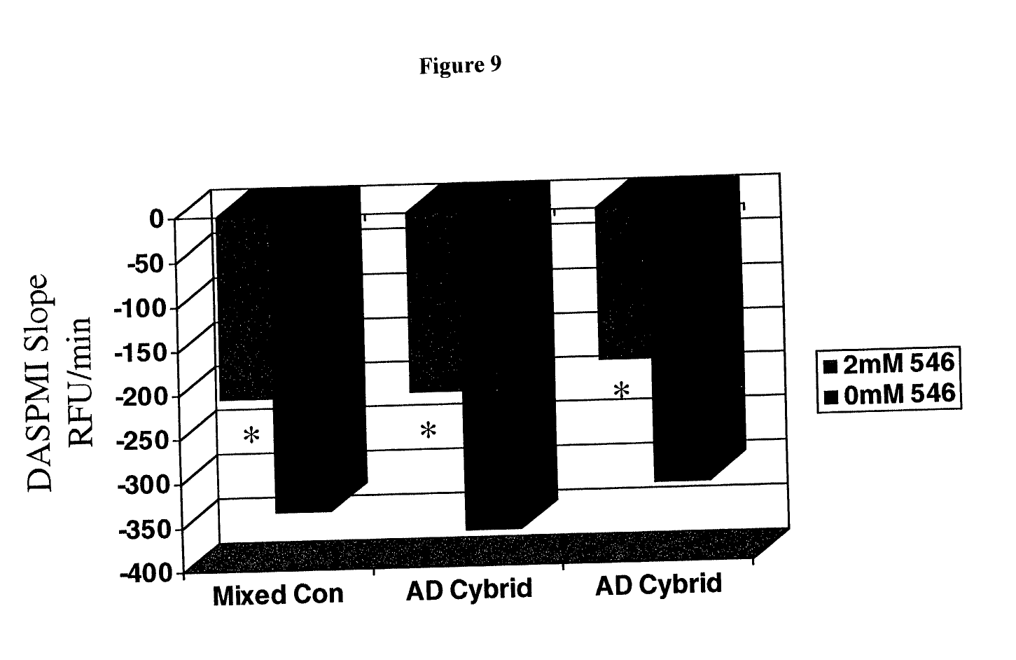

The assay for MPT by monitoring DASPMI fluorescence loss rate following induction of MPT was performed using two different AD cybrid cell lines and a control cybrid cell line as described in Example 1, with the following exceptions: Some groups of cultured cybrid cells were exposed to 2 mM 1-phenylbiguanide (compound “I”, RBI, Natick, Mass.) diluted in buffer or medium or to a vehicle control, for 20 min prior to MPT induction with 1 μM ionomycin. As shown in FIG. 9, 1-phenylbiguanide significantly (p<0.001, ANOVA as described supra) decreased the rate of loss of mitochondrial membrane potential following ionomycin induced MPT in all three cybrid cell lines.

### Example 9

### Identification of Agent that Regulates Apoptosis by Monitoring Caspase-3 Activation Following Ionomycin Induced MPT

Control SH-SY5Y cells, and control (normal) and AD cybrids produced from SH-SY5Y cells were as described above, and were induced to undergo MPT as described in Example 1. Some cultured cells were pretreated with 1-phenylbiguanide (I) as described in Example 8. Briefly, SH-SY5Y neuroblastoma cells (1×10⁵ cells) were rinsed with one volume 1×PBS, and then treated with 10 μM ionomycin (Calbiochem, San Diego, Calif.) in DMEM supplemented with 10% fetal calf serum (FCS) (Gibco, Life Technologies, Grand Island, N.Y.) for 10 minutes, followed by two washes with DMEM (10% FCS). After 6 h incubation at 37° C. in DMEM with 10% FCS, cells were visualized by light microscopy (200×magnification) to detect characteristic changes in cellular morphology associated with apoptotic cells.

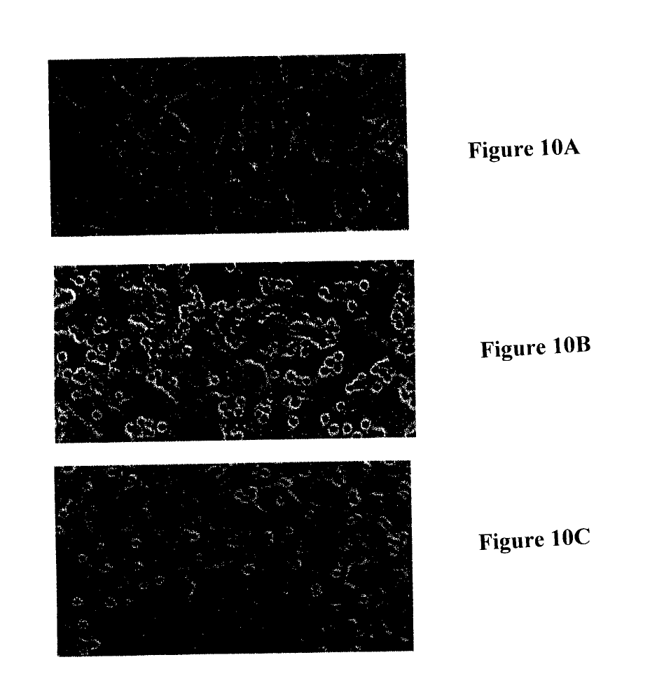

The results using parental SH-SY5Y cells are illustrated in FIG. 10. The normal appearance of these cells prior to induction of MPT is shown in FIG. 10A. After exposure to 10 μM ionomycin for four hours, approximately 80% of ionomycin treated cells exhibited membrane blebbing (FIG. 10B), indicative of entry by those cells into a final stage of apoptosis, compared to negligible apoptosis morphology (<5%) in untreated cells (not shown). Cells that were pretreated with (I) also exhibited substantially reduced apoptosis morphology (about 10-15% of cells; FIG. 10C ).

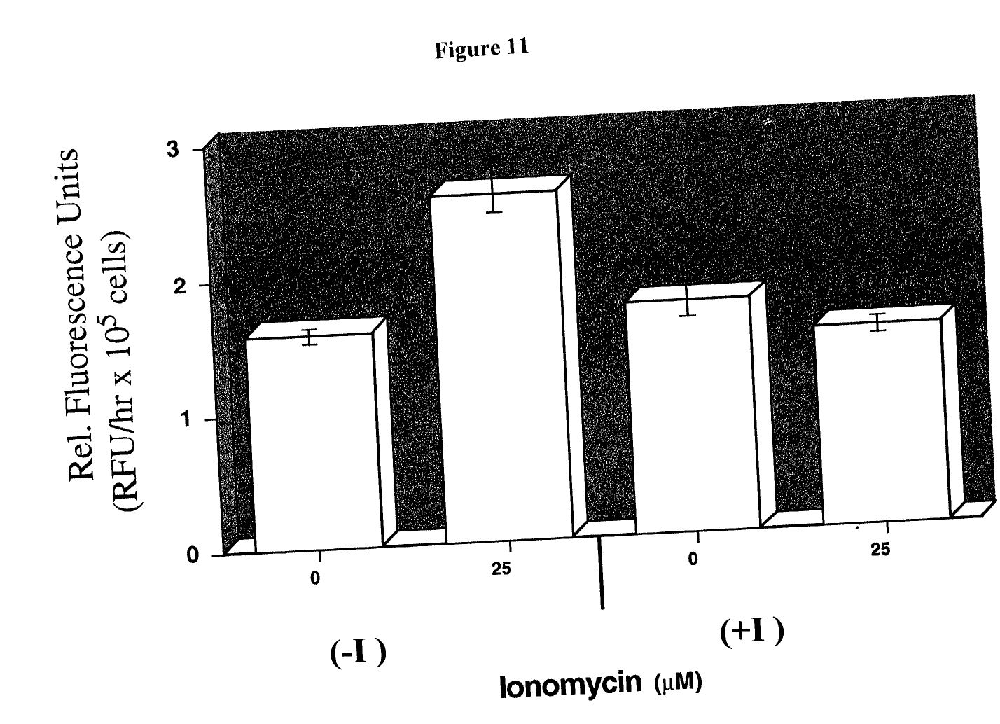

The effect of (I) on induction of the apoptosis-associated caspase-3 activity following ionomycin induced MPT was also assessed; as shown by its effect on DASPMI loss rate in Example 8, this agent inhibits MPT. Cells (AD cybrids or MixCon cybrids), cell culture, MPT induction and determination of caspase-3 activity were as described in Example 6, except that MPT was induced by 25 μM ionomycin and indicated cultures were pretreated with (I) as described in Example 8. The results, shown in FIG. 11 demonstrate that MPT induction by ionomycin induces significant caspase-3 activity in these cells, and that this induction of caspase-3 activity is inhibited in cells pretreated with the MPT inhibitor 1 -phenylbiguanide.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of identifying an agent that affects cell death comprising:
(a) contacting a first biological sample from a biological source with a candidate agent, wherein said first biological sample contains mitochondria;
(b) inducing cell death in said first biological sample and in a second biological sample from said biological source, wherein said second biological sample contains mitochondria;
(c) monitoring mitochondrial permeability transition in each of said first and second biological samples; and
(d) comparing mitochondrial permeability transition in said first biological sample to mitochondrial permeability transition in said second biological sample to detect a difference in mitochondrial permeability transition in said first biological sample relative to mitochondrial permeability transition in said second biological sample, and therefrom identifying an agent that affects cell death.

2. The method of claim 1 wherein said cell death is apoptosis.

3. The method of claim 1 wherein said cell death is necrosis.

4. The method of claim 1 wherein said first biological sample and said second biological sample are from a biological source having or suspected of being at risk for having a disease associated with altered mitochondrial function.

5. The method of claim 4 wherein said disease is selected from the group consisting of Alzheimer's Disease; diabetes mellitus; Parkinson's Disease; Huntington's disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome.

6. The method of claim 1 wherein step (b) comprises contacting said first biological sample and said second biological sample with a compound that increases Ca²⁺ concentrations in said mitochondria.

7. The method of claim 1 wherein step (b) comprises contacting said first biological sample and said second biological sample with a compound that binds a mitochondrial component.

8. The method of either claim 6 or claim 7 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

9. The method of either claim 6 or claim 7 wherein said compound binds a mitochondrial component and is selected from the group consisting of atractyloside and bongkrekic acid.

10. The method of claim 1 wherein step (b) comprises contacting said first biological sample and said second biological sample with a first compound that increases mitochondrial Ca²⁺ concentration and a second compound that binds a mitochondrial component.

11. The method of claim 1 wherein step (c) comprises contacting said samples with a detectable compound that accumulates in functioning mitochondria and that provides a detectable signal proportional to mitochondrial membrane potential.

12. The method of claim 11 wherein the detectable compound is selected from the group consisting of tetraphenylphosphonium ion; 2-,4-dimethylaminostyryl-N-methyl pyridinium; tetramethylrhodamine methyl ester; tetramethylrhodamine ethyl ester; rhodamine 123; 5,5′,6,6′-tetrachloro-1,1′,3,3′-tetraethylbezimidazolcarbocyanine iodide (JC-1); rhodamine 800; DiOC₆(3), rhodamine B hexyl ester and rhod-2.

13. A method of inhibiting apoptosis comprising contacting cells with 1-phenylbiguanide prior to or during said apoptosis.

14. A pharmaceutical composition comprising 1-phenylbiguanide.

15. The pharmaceutical composition of claim 14, further comprising a suitable carrier.

16. A method of identifying an agent that regulates mitochondrial permeability transition comprising:
a) contacting a first biological sample from a biological source with a candidate agent, wherein said first biological sample is a biological sample containing mitochondria;
b) inducing mitochondrial permeability transition in said first biological sample and in a second biological sample from said biological source, wherein said second biological sample is a biological sample containing mitochondria;
c) measuring mitochondrial membrane permeability in each of said first and second biological samples; and
d) comparing the amount of mitochondrial membrane permeability in the first biological sample to the amount of mitochondrial membrane permeability in the second biological sample to detect an effect of the candidate agent on mitochondrial membrane permeability, and therefrom determining suitability of the agent for treatment of a patient having a disease associated with altered mitochondrial function.

17. A method of identifying an agent suitable for treatment of a disease associated with altered mitochondrial function, comprising:
a) identifying a candidate agent that binds to a mitochondrial molecular component;
b) contacting a first biological sample containing mitochondria from a biological source with said candidate agent, wherein said first biological sample is a biological sample containing mitochondria;
c) inducing mitochondrial permeability transition in said first biological sample and in a second biological sample containing mitochondria from said biological source, wherein said second biological sample is a biological sample containing mitochondria;
d) measuring mitochondrial membrane permeability in each of said first and second biological samples; and
e) comparing the amount of mitochondrial membrane permeability in the first biological sample to the amount of mitochondrial membrane permeability in the second biological sample to detect an effect of the candidate agent on mitochondrial membrane permeability, and therefrom determining suitability of the agent for treatment of a patient having a disease associated with altered mitochondrial function.

18. The method of claim 17 wherein the mitochondrial molecular component is selected from the group consisting of an adenine nucleotide translocator, an electron transport chain component, a voltage dependent anion channel protein, a mitochondrial calcium uniporter, a mitochondrial associated hexokinase, a peripheral benzodiazepine receptor, a mitochondrial intermembrane creatine kinase, cyclophilin D and a Bcl-2 gene family encoded polypeptide.

19. The method of either claim 16 or claim 17 wherein the biological source is a cybrid cell.

20. The method of either claim 16 or claim 17 wherein mitochondrial permeability transition is induced by atractyloside.

21. The method of either claim 16 or claim 17 wherein mitochondrial permeability transition is induced by bongkrekic acid.

22. The method of either claim 16 or claim 17 wherein the disease associated with altered mitochondrial function is selected from the group consisting of Alzheimer's Disease; diabetes mellitus; Parkinson's Disease; Huntington's disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome.

23. The method of either claim 16 or claim 17 wherein the disease associated with altered mitochondrial function is Alzheimer's Disease.

24. The method of either claim 16 or claim 17 wherein said first biological sample and said second biological sample are from a biological source having or suspected of being at risk for having a disease associated with altered mitochondrial function.

25. The method of either claim 16 or claim 17 wherein the step of inducing mitochondrial permeability transition comprises contacting said first biological sample and said second biological sample with a compound that increases Ca²⁺ concentrations in said mitochondria.

26. The method of claim 25 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

27. The method of either claim 16 or claim 17 wherein the step of inducing mitochondrial permeability transition comprises contacting said first biological sample and said second biological sample with a compound that binds a mitochondrial component.

28. The method of claim 27 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

29. The method of either claim 16 or claim 17 wherein the step of inducing mitochondrial permeability transition comprises contacting said first and second biological samples with an apoptogen.

30. The method of either claim 16 or claim 17 wherein the step of inducing mitochondrial permeability transition comprises contacting each of said first biological sample and said second biological sample with a first compound that increases mitochondrial Ca²⁺ concentration and with a second compound that binds a mitochondrial component.

31. The method of claim 30 wherein each of said first and second compounds is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

32. The method of either claim 16 or claim 17 wherein the step of measuring mitochondrial permeability comprises detecting an indicator of inner mitochondrial membrane potential.

33. The method of claim 32 wherein the indicator of inner mitochondrial membrane potential is selected from the group consisting of tetraphenylphosphonium ion; 2-,4-dimethylaminostyryl-N-methyl pyridinium; tetramethylrhodamine methyl ester; tetramethylrhodamine ethyl ester; rhodamine 123; 5,5′,6,6′-tetrachloro-1,1′,3,3′-tetraethylbezimidazolcarbocyanine iodide (JC-1); rhodamine 800; DiOC₆(3), rhodamine B hexyl ester and rhod-2.

34. The method of either claim 16 or claim 17 wherein the step of measuring mitochondrial permeability transition comprises detecting apoptosis.

35. The method of claim 34 wherein detecting apoptosis is selected from the group consisting of detecting altered cellular morphology, detecting DNA fragmentation, detecting translocation of phosphatidylserine to a plasma membrane outer leaflet, detecting induction of one or more caspase activities and detecting the release of cytochrome c from said mitochondria.

36. A method of inhibiting mitochondrial permeability transition in cells comprising contacting said cells with 1-phenylbiguanide prior to or during said mitochondrial permeability transition.

37. A method of identifying an agent that affects electron transport chain activity in mitochondria comprising:
(a) contacting a first sample from a biological source with a candidate agent, wherein said first sample contains mitochondria;
(b) inducing mitochondrial permeability transition in said first biological sample and in a second biological sample from said biological source, wherein said second sample contains mitochondria;
(c) monitoring mitochondrial permeability transition in each of said first and second samples; and
(d) comparing mitochondrial permeability transition in said first biological sample to mitochondrial permeability transition in said second biological sample to detect a difference in mitochondrial permeability transition in said first biological sample relative to mitochondrial permeability transition in said second biological sample, and therefrom identifying an agent that affects electron transport chain activity.

38. A method of identifying an agent suitable for treatment of a patient having a disease associated with altered mitochondrial function, comprising:
a) contacting a first biological sample from a biological source with a candidate agent, wherein said first biological sample is a biological sample containing mitochondria;
b) inducing mitochondrial permeability transition in said first biological sample and in a second biological sample from said biological source, wherein said second biological sample is a biological sample containing mitochondria;
c) measuring mitochondrial membrane permeability in each of said first and second biological samples; and
d) comparing the amount of mitochondrial membrane permeability in the first biological sample to the amount of mitochondrial membrane permeability in the second biological sample to detect an effect of the candidate agent on mitochondrial membrane permeability, and therefrom determining suitability of the agent for treatment of a patient having a disease associated with altered mitochondrial function.

39. A method for detecting a risk or presence of a disease associated with altered mitochondrial function in a subject, comprising:
a) inducing mitochondrial permeability transition in a first biological sample and in a second biological sample, wherein
said first biological sample contains mitochondria and is from a first subject suspected of having or being at risk for having a disease associated with altered mitochondrial function, and wherein
said second biological sample contains mitochondria and is from a second subject known to be free of a risk or presence of a disease associated with altered mitochondrial function;
b) measuring mitochondrial membrane permeability in each of said first and second biological samples; and
c) comparing the amount of mitochondrial membrane permeability in the first biological sample to the amount of mitochondrial membrane permeability in the second biological sample, and therefrom determining a risk or presence of a disease associated with altered mitochondrial function in said first subject.

40. The method of claim 39 wherein mitochondrial permeability transition in the first biological sample is induced in a cybrid cell having mitochondria from said first subject.

41. The method of claim 39 wherein mitochondrial permeability transition in the second biological sample is induced in a cybrid cell having mitochondria from said second subject.

42. The method of claim 41 wherein mitochondria from the second subject are derived from a plurality of subjects known to be free of a risk or presence of a disease associated with altered mitochondrial function.

43. The method of claim 39 wherein the step of inducing mitochondrial permeability transition comprises contacting said first biological sample and said second biological sample with a compound that increases Ca²⁺ concentrations in said mitochondria.

44. The method of claim 43 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

45. The method of claim 39 wherein the step of inducing mitochondrial permeability transition comprises contacting said first biological sample and said second biological sample with a compound that binds a mitochondrial component.

46. The method of claim 45 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

47. The method of claim 39 wherein the step of inducing mitochondrial permeability transition comprises contacting said first and second biological samples with an apoptogen.

48. The method of claim 39 wherein the step of inducing mitochondrial permeability transition comprises contacting each of said first biological sample and said second biological sample with a first compound that increases mitochondrial Ca²⁺ concentration and with a second compound that binds a mitochondrial component.

49. The method of claim 48 wherein each of said first and second compounds is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

50. The method of claim 39 wherein the step of measuring mitochondrial permeability comprises detecting an indicator of inner mitochondrial membrane potential.

51. The method of claim 50 wherein the indicator of inner mitochondrial membrane potential is selected from the group consisting of tetraphenylphosphonium ion; 2-,4-dimethylaminostyryl-N-methyl pyridinium; tetramethylrhodamine methyl ester; tetramethylrhodamine ethyl ester; rhodamine 123; 5,5′,6,6′-tetrachloro-1,1′,3,3′-tetraethylbezimidazolcarbocyanine iodide (JC-1); rhodamine 800; DiOC₆(3), rhodamine B hexyl ester and rhod-2.

52. The method of claim 39 wherein the step of measuring mitochondrial permeability transition comprises detecting apoptosis.

53. The method of claim 52 wherein detecting apoptosis is selected from the group consisting of detecting altered cellular morphology, detecting DNA fragmentation, detecting translocation of phosphatidylserine to a plasma membrane outer leaflet, detecting induction of one or more caspase activities and detecting the release of cytochrome c from said mitochondria.

54. The method of claim 39 wherein the disease associated with altered mitochondrial function is selected from the group consisting of Alzheimer's Disease; diabetes mellitus; Parkinson's Disease; Huntington's disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome.

55. The method of claim 39 wherein the disease associated with altered mitochondrial function is Alzheimer's Disease.

56. A method for identifying a mitochondrial molecular component that regulates mitochondrial permeability transition, comprising:
a) identifying a candidate agent that alters mitochondrial membrane permeability by
(i) contacting a first biological sample from a biological source with said candidate agent, wherein said first biological sample is a biological sample containing mitochondria;
(ii) inducing mitochondrial permeability transition in said first biological sample and in a second biological sample from said biological source, wherein said second biological sample is a biological sample containing mitochondria;
(iii) measuring mitochondrial membrane permeability in each of said first and second biological samples; and
(iv) comparing the amount of mitochondrial membrane permeability in the first biological sample to the amount of mitochondrial membrane permeability in the second biological sample to determine an effect of the candidate agent on mitochondrial membrane permeability; and
b) contacting the candidate agent with a plurality of mitochondrial molecular components under conditions and for a time sufficient to permit detectable binding of the candidate agent to at least one mitochondrial molecular component, and therefrom identifying a mitochondrial molecular component that regulates mitochondrial permeability transition.

57. The method of claim 56 wherein the biological source comprises a cybrid cell.

58. The method of claim 57 wherein the cybrid cell comprises mitochondria derived from a subject having a disease associated with altered mitochondrial function.

59. The method of claim 58 wherein the disease associated with altered mitochondrial function is selected from the group consisting of Alzheimer's Disease; diabetes mellitus; Parkinson's Disease; Huntington's Disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome.

60. The method of claim 58 wherein the disease associated with altered mitochondrial function is Alzheimer's Disease.

61. The method of claim 56 wherein the step of inducing mitochondrial permeability transition comprises contacting said first biological sample and said second biological sample with a compound that increases Ca²⁺ concentrations in said mitochondria.

62. The method of claim 61 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

63. The method of claim 56 wherein the step of inducing mitochondrial permeability transition comprises contacting said first biological sample and said second biological sample with a compound that binds a mitochondrial component.

64. The method of claim 63 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

65. The method of claim 56 wherein the step of inducing mitochondrial permeability transition comprises contacting said first and second biological samples with an apoptogen.

66. The method of claim 56 wherein the step of inducing mitochondrial permeability transition comprises contacting each of said first biological sample and said second biological sample with a first compound that increases mitochondrial Ca²⁺ concentration and with a second compound that binds a mitochondrial component.

67. The method of claim 66 wherein each of said first and second compounds is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

68. The method of claim 56 wherein the step of measuring mitochondrial permeability comprises detecting an indicator of inner mitochondrial membrane potential.

69. The method of claim 68 wherein the indicator of inner mitochondrial membrane potential is selected from the group consisting of tetraphenylphosphonium ion; 2-,4-dimethylaminostyryl-N-methyl pyridinium; tetramethylrhodamine methyl ester; tetramethylrhodamine ethyl ester; rhodamine 123; 5,5′,6,6′-tetrachloro-1,1′,3,3′-tetraethylbezimidazolcarbocyanine iodide (JC-1); rhodamine 800; DiOC₆(3), rhodamine B hexyl ester and rhod-2.

70. The method of claim 56 wherein the step of measuring mitochondrial permeability transition comprises detecting apoptosis.

71. The method of claim 70 wherein detecting apoptosis is selected from the group consisting of detecting altered cellular morphology, detecting DNA fragmentation, detecting translocation of phosphatidylserine to a plasma membrane outer leaflet, detecting induction of one or more caspase activities and detecting the release of cytochrome c from said mitochondria.

72. The method of claim 59 wherein binding of the mitochondrial molecular component to the agent is determined by affinity isolation of the mitochondrial molecular component.

73. The method of claim 59 wherein binding of the mitochondrial molecular component to the agent is determined by affinity labeling of the mitochondrial molecular component.

74. The method of claim 59 wherein binding of the agent to the mitochondrial molecular component is determined following expression of a nucleic acid library encoding said mitochondrial molecular component.

75. A method for determining a risk for or presence of Alzheimer's disease in a subject, comprising:
a) inducing mitochondrial permeability transition in a first biological sample from a first subject suspected of having or being at risk for having Alzheimer's disease and in a second biological sample from a second subject known to be free of having or being at risk for having Alzheimer's disease, wherein said first and second biological samples are biological samples containing mitochondria;
b) measuring mitochondrial membrane permeability in each of said first and second biological samples;
c) determining the apolipoprotein E genotype of each of said first and second subjects; and
d) correlating the amount of mitochondrial membrane permeability in each of the first and second biological samples with the apolipoprotein E genotype of each of said first and second subjects, and therefrom determining a risk for or presence of Alzheimer's disease in the first subject.

76. The method of claim 75 wherein mitochondrial permeability transition in the first biological sample is induced in a cybrid cell having mitochondria from said first subject.

77. The method of claim 75 wherein mitochondrial permeability transition in the second biological sample is induced in a cybrid cell having mitochondria from said second subject.

78. The method of claim 77 wherein mitochondria from the second subject are derived from a plurality of subjects known to be free of having or being at risk for having Alzheimer's disease.

79. The method of claim 75 wherein the step of inducing mitochondrial permeability transition comprises contacting said first biological sample and said second biological sample with a compound that increases Ca²⁺ concentrations in said mitochondria.

80. The method of claim 79 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

81. The method of claim 75 wherein the step of inducing mitochondrial permeability transition comprises contacting said first biological sample and said second biological sample with a compound that binds a mitochondrial component.

82. The method of claim 81 wherein said compound is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

83. The method of claim 75 wherein the step of inducing mitochondrial permeability transition comprises contacting said first and second biological samples with an apoptogen.

84. The method of claim 75 wherein the step of inducing mitochondrial permeability transition comprises contacting each of said first biological sample and said second biological sample with a first compound that increases mitochondrial Ca²⁺ concentration and with a second compound that binds a mitochondrial component.

85. The method of claim 84 wherein each of said first and second compounds is selected from the group consisting of thapsigargin, an amino acid neurotransmitter, glutamate, N-methyl-D-aspartic acid, carbachol, an ionophore, ionomycin, an apoptogen, atractyloside and bongkrekic acid.

86. The method of claim 75 wherein the step of measuring mitochondrial permeability comprises detecting an indicator of inner mitochondrial membrane potential.

87. The method of claim 86 wherein the indicator of inner mitochondrial membrane potential is selected from the group consisting of tetraphenylphosphonium ion; 2-,4-dimethylaminostyryl-N-methyl pyridinium; tetramethylrhodamine methyl ester; tetramethylrhodamine ethyl ester; rhodamine 123; 5,5′,6,6′-tetrachloro-1,1′,3,3′-tetraethylbezimidazolcarbocyanine iodide (JC-1); rhodamine 800; DiOC₆(3), rhodamine B hexyl ester and rhod-2.

88. The method of claim 75 wherein the step of measuring mitochondrial permeability transition comprises detecting apoptosis.

89. The method of claim 88 wherein detecting apoptosis is selected from the group consisting of detecting altered cellular morphology, detecting DNA fragmentation, detecting translocation of phosphatidylserine to a plasma membrane outer leaflet, detecting induction of one or more caspase activities and detecting the release of cytochrome c from said mitochondria.

90. The method of any one of claims 34, 52, 70 or 88 wherein apoptosis is detected by measuring induction of a caspase protease activity that cleaves a polypeptide substrate.

91. The method of claim 90 wherein the caspase protease activity is selected from the group consisting of caspase-1 protease activity and caspase-3 protease activity.

92. The method of claim 91 wherein the caspase protease activity is caspase-1 protease activity.

93. The method of claim 91 wherein the caspase protease activity is caspase-3 protease activity.

94. The method of claim 91 wherein the polypeptide substrate is selected from the group consisting of Asp-Glu-Val-Asp-AMC and Tyr-Val-Ala-Asp-Z.

95. The method of any one of claims 34, 52, 70 or 88 wherein apoptosis is detected by determining the presence of cytochrome c released from mitochondria.

96. The method of claim 95, comprising determination of released cytochrome c by binding to an antibody specific for cytochrome c.

97. The method of claim 96, further comprising determining the molecular mass of released cytochrome c that binds to an antibody specific for cytochrome c by matrix assisted laser desorption ionization time-of-flight mass spectrometry.

98. A method of identifying an agent that regulates mitochondrial function in a species-specific manner comprising:
(a) contacting a first biological sample with a candidate agent, wherein said first biological sample contains mitochondria and is from a biological source organism of a first species;
(b) inducing mitochondrial permeability transition in said first sample and in a second biological sample, wherein said second sample contains mitochondria and is from an organism of a second species;
(c) monitoring mitochondrial permeability transition in each of said first and second biological samples; and
(d) comparing mitochondrial permeability transition in said first biological sample to mitochondrial permeability transition in said second biological sample to detect a difference in mitochondrial permeability transition in said first biological sample relative to mitochondrial permeability transition in said second biological sample, and therefrom identifying an agent that regulates mitochondrial function in a species specific manner.

99. The method of claim 98 wherein said first species is Homo sapiens and said second species is a eukaryotic pathogen or parasite of Homo sapiens.

100. An agent identified according to the method of claim 99.

101. The method of claim 98 wherein said first species is an undesired insect species and said second species is a desired insect species.

102. An agent identified according to the method of claim 101.

103. The method of claim 98 wherein said first species is an desired plant species and said second species is an undesired plant species or an undesired insect species.

104. The method of claim 103 wherein said undesired insect species is a member of the phylum Lepidoptera.

105. An agent identified according to the method of claim 104.

106. A method of identifying a genotype associated with a disease comprising:
(a) contacting a first biological sample from a biological source with a candidate agent, wherein said first biological sample contains mitochondria;
(b) inducing cell death in said first biological sample and in a second biological sample from said biological source, wherein said second biological sample contains mitochondria;
(c) monitoring mitochondrial permeability transition in each of said first and second biological samples; and
(d) comparing mitochondrial permeability transition in said first biological sample to mitochondrial permeability transition in said second biological sample to detect a difference in mitochondrial permeability transition in said first biological sample relative to mitochondrial permeability transition in said second biological sample, and therefrom identifying a genotype associated with the disease.

107. The method of claim 106 wherein said disease is selected from the group consisting of Alzheimer's disease, diabetes mellitus, Parkinson's disease, Huntington's disease, dystonia, Leber's hereditary optic neuropathy, mitochondrial encephalopathy, lactic acidosis, schizophrenia and myodegenerative disorders such as MELAS and MERRF.

108. A method of treating a disease associated with altered mitochondrial function, comprising administering a composition that regulates mitochondrial permeability transition.
